SEQUENCE LISTING (13 sequences)

SEQ ID NO: 1 - RNA, 23 nt, Homo sapiens
  source 1..23: mol_type = other RNA
tacagcaggcacagacaggcagt

SEQ ID NO: 2 - RNA, 22 nt, Homo sapiens
  source 1..22: mol_type = other RNA
caggaaggagacaggcagttca

SEQ ID NO: 3 - RNA, 22 nt, Homo sapiens
  source 1..22: mol_type = other RNA
tctatacagaccctggcttttc

SEQ ID NO: 4 - RNA, 22 nt, Homo sapiens
  source 1..22: mol_type = other RNA
gggagccaggaagtattgatgt

SEQ ID NO: 5 - RNA, 23 nt, Homo sapiens
  source 1..23: mol_type = other RNA
taggtggcgccggaggagtcatt

SEQ ID NO: 6 - RNA, 21 nt, Homo sapiens
  source 1..21: mol_type = other RNA
tcttgtgttctctagatcagt

SEQ ID NO: 7 - RNA, 22 nt, Homo sapiens
  source 1..22: mol_type = other RNA
actgcattatgagcacttaaag

SEQ ID NO: 8 - RNA, 17 nt, Homo sapiens
  source 1..17: mol_type = other RNA
ggggctgggcgcgcgcc

SEQ ID NO: 9 - RNA, 22 nt, Homo sapiens
  source 1..22: mol_type = other RNA
aatcactaaccacacggccagg

SEQ ID NO: 10 - RNA, 21 nt, Homo sapiens
  source 1..21: mol_type = other RNA
tgattgtccaaacgcaattct

SEQ ID NO: 11 - RNA, 22 nt, Homo sapiens
  source 1..22: mol_type = other RNA
aggggactggttaatagaacta

SEQ ID NO: 12 - RNA, 22 nt, Homo sapiens
  source 1..22: mol_type = other RNA
atcaaggatcttaaactttgcc

SEQ ID NO: 13 - RNA, 23 nt, Homo sapiens
  source 1..23: mol_type = other RNA
cagtgcaatagtattgtcaaagc

(19) United States
(12) Patent Application Publication
(10) Pub. No.: US 2026/0240915 A1
(43) Pub. Date: Aug. 20, 2026

(54) USE OF EXTRACELLULAR VESICLES FOR THE TREATMENT OF CANCER

(71) Applicant: HENRY FORD HEALTH SYSTEM, Detroit, MI (US)

(72) Inventors: Michael CHOPP, Southfield, MI (US); Dilip K. MOONKA, Beverly Hills, MI (US); Zhenggang ZHANG, Troy, MI (US); Louie SEMAAN, Detroit, MI (US)

(21) Appl. No.: 19/161,401

(22) PCT Filed: Mar. 1, 2024
(86) PCT No.: PCT/US2024/018058

§ 371 Date: Sep. 2, 2025

Related U.S. Application Data
(60) Provisional application No. 63/488,280, filed on Mar. 3, 2023.

(51) Int. Cl. A61K 35/44; A61K 31/44; A61K 31/7105; A61K 33/243; A61K 45/06; A61P 35/00; C12N 15/113

(52) U.S. Cl. CPC A61K 35/44; A61K 31/44; A61K 31/7105; A61K 33/243; A61K 45/06; A61P 35/00; C12N 15/113; C12N 2310/141

(57) ABSTRACT

Some embodiments comprise a method and kit for the treatment and prevention of a cancer the method including the steps of administering a therapeutically effective amount of stressed mammalian extracellular vesicles. In some embodiments, the method may further include administration of at least one adjunct treatment comprising a chemotherapeutic agent, surgical resection or radiation therapy to a subject in need thereof. A kit for the treatment and prevention of a cancer composes a therapeutically effective combination of stressed mammalian exosomes, and at least one of: a chemotherapeutic agent, a dose of a radioactive agent for performing radiation therapy and a surgical device for performing resection surgery of a tumor.

### SEQUENCE LISTING

This application incorporates by reference in its entirety the Sequence Listing entitled “SequenceListing.xml” (12 KB), which was created on 27 Feb. 2024 at 4:25 PM.

### TECHNICAL FIELD

Without limitation, some embodiments comprise methods, systems, and compositions relating to the treatment of a broad array of cancers, with a therapeutically effective amount of mammalian extracellular vesicles (i.e. extracellular vesicles) that are derived from mammalian cells that have been stressed under conditions of hypoxia and/or oxygen and glucose deprivation (OGD).

### BACKGROUND

Primary liver cancer is currently the fifth most common cause of cancer deaths among men, and ninth among women in the US, with the numbers increasing yearly. The most recent data indicate that in 2008 there were an estimated 21,370 new cases of liver and bile duct cancer of which the majority are hepatocellular carcinomas (HCCs), with 18,410 deaths (Institute, N.C., SEER Cancer Statistics Review, 1975-2005, Ries L A G, et al., Editors. 2008.). Worldwide, it is the fourth most common cancer, with approximately 663,00 fatal cases reported in 2008; based on current trends and baseline models, the incidence is expected to rise to 756,000 in 2015, and 955,000 in 2030 (Mathers, C. D. and D. Loncar, Projections of global mortality and burden of disease from 2002 to 2030. PLOS Med, 2006. 3, 11, p. e442.). Although it is comparatively uncommon in the US, its incidence has been rising over the last 20 years partially as a result of burgeoning numbers of cases of chronic hepatitis C (Caldwell, S. and S. H. Park, The epidemiology of hepatocellular cancer: from the perspectives of public health problem to tumor biology. J Gastroenterol, 2009. 44 Suppl 19: p. 96-101. El-Serag, H. B., et al., The continuing increase in the incidence of hepatocellular carcinoma in the United States: an update. Ann Intern Med, 2003. 139 (10): p. 817-23) one of the principal causes along with hepatitis B and aflatoxin exposure.

HCC primarily occurs in patients with cirrhosis which causes significant liver dysfunction and limited hepatic reserve. As a result of this and because liver cancers are often advanced at the time of diagnosis, only 10-15% of patients with HCC are candidates for curative surgery. For the majority of HCC patients, systemic chemotherapies or supportive therapies are the mainstay treatment options. Nevertheless, most chemotherapeutic agents show limited effectiveness and have not been able to improve patient survival. See e.g., Yeo W. et al., J. of the National Cancer Institute 97:1532-8 (2005), Gish R. G. et al., J. of Clinical Oncology 25:3069-75 (2007), Ramanathan R. K. et al., J. of Clinical Oncology 24:4010 (2006), and O'Dwyer P. J. et al., J. of Clinical Oncology 24:4143 (2006). A recent Phase III randomized trial of Sorafenib, an oral multi-kinase inhibitor of the VEGF receptor, PDGF receptor, and Raf, on hepatitis B-related HCC patients showed for the first time to prolong survival of advanced stage patients. See Cheng A. L. et al., Lancet Oncology 10:25-34 (2009). However, the median overall survival only increased from 4.2 months in the placebo group to 6.5 months in the treatment group. An additional study of 600 patents, demonstrated increases survival of three months with Sorafenib (Llovet NEJM 2008). Moreover, HCC is frequently chemotherapy-resistant and known to over-express multi-drug resistance genes, such as MDR1 (P-gp) and the multi-drug resistance proteins (MRPs). See e.g., Ng I. et al., American J. of Clinical Pathology 113:355-63 (2000), Endicott J. A. et al., Annual Review of Biochemistry 58:137-71 (1989), and Park J. G. et al., J. of the National Cancer Institute 86:700-5 (1994). The adverse clinical course of most HCC patients underscores much need for more efficacious chemotherapies and development of targeting strategies. Various embodiments of the present disclosure address and resolve these pressing needs. While PD-1 checkpoint inhibitors, including atezolizumab, can be effective in the treatment of HCC, they are effective in a minority of patients and are not suitable for patients with severely decompensated liver function. There remains a compelling need for additional effective cancer therapies.

Cancer treatment remains challenging with limited options also for glioblastoma multiforme and pancreatic and lung cancer, among others. There is a long felt need for therapeutic regimens that are both disease-modifying and effective in treating patients with cancer. The present disclosure addresses the need for therapeutic treatments that can be administered as a stand-alone therapy or be combined with existing standards of care in the treatment of cancers, in particular as therapies for human cancers and tumors.

### SUMMARY

In a first aspect, the present disclosure provides methods for treating and/or preventing a cancer in a subject suspected of having a cancer or diagnosed with a cancer. For example, in a human subject, the method comprising: administering a therapeutically effective amount of stressed mammalian extracellular vesicles derived from hypoxic or oxygen and glucose deprived mammalian cells, to the subject with cancer or suspected of having cancer.

In another aspect, the present disclosure provides a kit comprising mammalian exosomes, a dose of a chemotherapeutic agent, and a package insert comprising instructions for using the mammalian stressed extracellular vesicles and chemotherapeutic agent in combination to treat a subject with cancer.

As used herein the stressed mammalian extracellular vesicles can be used to prevent and/or treat a variety of cancers. In advantageous embodiments, the extracellular vesicles include exosomes, which may be used to prevent and/or treat a number of different cancer types, including, but not limited to: breast cancer (e.g. ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), thyroid cancer, endocrine system cancer, brain cancer, cervical cancer, colon cancer, head & neck cancer, liver cancer, kidney cancer, non-small cell lung cancer, mesothelioma, stomach cancer, uterine cancer, Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, prostate cancer, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, undifferentiated cell leukemia, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum, hepatocellular carcinoma (HCC), intrahepatic cholangiocarcinoma, angiosarcoma and hemangiosarcoma, hepatoblastoma, hemangiomas, hepatic adenomas, focal nodular hyperplasia, pancreatic adenocarcinoma, adenosquamous carcinomas, squamous cell carcinomas, signet ring cell carcinomas, undifferentiated carcinomas, carcinoma of the ampulla of Vater, undifferentiated carcinomas with giant cells, serous cystic neoplasms, mucinous cystic neoplasms, intraductal papillary mucinous neoplasms, and solid pseudopapillary neoplasms and secondary cancers such as metastatic cancer or refractory or advanced cancers.

### BRIEF DESCRIPTION OF DRAWINGS

Some embodiments will now be described, by way of example only and without waiver or disclaimer of other embodiments, with reference to the accompanying drawings, in which:

FIG. 1. The effect of OGD-Exos on the viability of liver cancer cells. Three 4 h-OGD-Exo concentrations (1×10⁷, 1×10⁸ and 1×10⁹ particles/ml) were used in this study. The 4 h-OGD-Exo reduced the viability of HepG2 and Hep3B hepatocellular carcinoma (HCC) lines in a dose dependent manner. At the highest concentration, 1×10⁹, 4 h-OGD-Exo reduced HCC cell viability by 51% compared to HCC cells treated with no exosomes at all. 0h-OGD-Exo had no effect on HCC viability and no exosomes had an effect on the benign THLE-2 liver cell line.

FIG. 2. The effect of 4 h-OGD-Exos on cell viability of multiple cancer cells. There are two exosome concentrations (1×10⁸ and 1×10⁹ particles (p)/ml) displayed in this figure. The number of dots represent individual experiments or wells. 4 h-OGD-Exo at concentrations of 10⁸ p/ml and 10⁹ p/ml are more effective than no exosomes or 0h-OGD-Exo across an array of cancer cell lines. No exosomes were active against the benign THLE-2 liver cell line and the multi-drug resistant ovarian cancer line A2780cis.

FIG. 3. The effect of 4 h-OGD-Exo alone or in combination with HCEC-m214-Exo on liver cancer cells. All exosomes were used at a concentration of 1×10⁸ p/ml. The combination of 4 h-OGD-Exo and HCEC-m214-Exo in red decreased HCC viability more than either OGD-Exo alone or HCEC-m214-Exo alone. No exosomes had an effect on the benign THLE-2 liver cells.

FIG. 4. The effect of 4 h-OGD-Exo alone or in combination with HCEC-m214-Exo across multiple cancer types. Exosomes were tested at concentrations of 10⁸ and 10⁹ p/ml. Again, 4 h-OGD-Exo are effective in reducing cancer viability across all tumor types tested with better efficacy seen with the higher dose. The combination of 4 h-OGD-Exo and HCEC-m214-Exo dark and light purple decreased HCC viability more than either exosome population alone. No exosomes had an effect on the benign THLE-2 liver cells or the NIH 3T3 fibroblast line. No exosomes are effective against the multi-drug resistant ovarian 2780cis line.

FIG. 5. The effect of OGD-Exo on patient liver cancer cells with chemotherapeutic agents. 4 h-OGD-Exo are effective in reducing cancer viability of patient liver cancer cells taken at the time of liver transplant. The OGD-Exo have no effect on normal patient liver cells taken from normal liver away from the HCC (Distal). This activity is enhanced in combination with chemotherapeutic agents and with mR214-Exo and chemotherapeutic agents. Oxaliplatin data is shown in FIG. 5A and Sorafenib data in FIG. 5B.

FIG. 6. The effect of Hypo-Exo from HCEC cells exposed to increasing hypoxia on liver cancer lines. HCEC cells were exposed to increasing lengths of hypoxia and the Hypo-Exo were tested against HCC cells at 10⁷ and 10⁸ p/ml. With 24 hours of hypoxia, Hypo-Exo at 10⁸ p/ml lead to decreased viability of HepG2 cells of 73% and Hep3B cells of 76%. Hypo-Exo had no effect on benign THLE-2 cells.

FIG. 7. The effect of Hypo-Exo on multiple non-hepatic cancer cell lines. 24 h-Hypo-Exos were tested against an array of non-hepatic cancer cell lines at 10⁸ and 10⁹ p/ml. 24 h-Hypo-Exo, at a dose of 10⁹ p/ml, led to a decrease in cell viability for PANC-1 (pancreas) of 64%, A549 (lung) of 76%, MDA-MD-468 (breast) of 86%, MDA-MD-231 (breast) of 77%, PC3 (prostate) of 78%, HCT-116 (colon) of 64%, A2780 (ovarian) of 65%, OVCAR (ovarian) of 64% and F2 (ovarian) of 56%. The exosomes again had no activity against the A2780cis (multi-resistant ovarian cancer line) or the benign NIH 3T3 (fibroblast) lines.

FIG. 8. The effect of m214-Hypo-Exo on the viability of HepG2 and Hep3B HCC cells and PANC-1 pancreatic cancer cells. For HepG2, Hep3B and PANC-1 cancer cells, 24 h-Hypo-Exo from HCEC cells overexpressing miR-214 (m214-Hypo-Exo), in green, were more potent in decreasing cancer cell viability than Hypo-Exo from HCEC cells not over-expressing miR-214. No exosomes had an effect on the non-cancerous THLE-2 liver cell line.

FIG. 9. The effect of 24 h-Hypo-Exo in combination with Sorafenib on the cell viability of HepG2, Hep3B and THLE-2 cells. The combination of Hypo-Exo and Sorafenib is more potent than either Hypo-Exo alone, Sorafenib alone or Sorafenib with 0h-Hypo-Exo.

FIG. 10. The effect of 24 h-Hypo-Exo derived from HUVECs, BM-MSCs, and Adi-MSCs on cell viability of multiple cancer cell lines. Panel A shows cell viability of THLE-2, HepG2 and Hep3B cells treated with 24 h-Hypo-Exo isolated from HCEC, HUVECs, BM-MSCs, and Adi-MSCs. Panel B shows cell viability of various cancer cells treated with 24 h-Hypo-Exo isolated from HUVECs, BM-MSCs, and Adi-MSCs. Only HCEC derived Hypo-Exo exhibit significant anti-cancer effect.

FIG. 11. The effect of CPZ and nystatin on cell viability of HepG2 and Hep3B. Panels A and B show the effect of different concentrations of CPZ (A) and Nystatin (B) on HepG2 and Hep3B cell viabilities. CPZ and nystatin blunt the anti-tumor effect of Hypo-Exo on HepG2 cells but nystatin blunts the effect of Hypo-Exo on Hep3B cells but not HepG2 cells.

FIG. 12. Protein profiles of Hypo-Exo vs. Naïve-Exo. A heatmap shows increased and reduced cargo proteins in Hypo-Exo and non-Hypo-Exo (naïve-Exo) from HCEC cells: n=3/group.

FIG. 13. Hypo-Exo cargo proteins and their functions. Panels A and B show increased (A) and reduced (B) proteins and their functions.

FIG. 14. Exosomal cargo miRNA profile. A volcano plot showed enriched and decreased miRNAs within Hypo-Exo compared to Naïve-Exo.

FIG. 15. Exosomal cargo miRNA profile. A bar graph showed enriched miRNAs within Hypo-Exo compared to Naïve-Exo using two different statistical packages.

FIG. 16. Exosomal cargo miRNA profile. A bar graph showed decreased miRNAs within Hypo-Exo compared to Naïve-Exo using two different statistical packages.

FIG. 17. Exosomal cargo miRNA profile. Pathway analysis demonstrating potential biological processes mediated by miRNA upregulated in Hypo-Exo vs. Naïve-Exo.

FIG. 18. The combination of Sorafenib with Hypo-Exo reduced CDK6 expression. Western blot analysis showed CDK6 levels in HepG2 and Hep3B cancer cells treated with or without Sorafenib and 24 h-Hypo-Exo or Naïve-Exo.

FIG. 19. Exosomal cargo miRNA profile. Pathway analysis demonstrating potential biological processes mediated by miRNA downregulated in Hypo-Exo vs. Naïve-Exo.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Each example and embodiment of the disclosure is to be applied mutatis mutandis to each and every other example or embodiment unless specifically stated otherwise.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any of said steps or features.

The present disclosure is not to be limited in scope by the specific examples described herein, which are intended for the purpose of exemplification only. Functionally-equivalent compositions and methods are clearly within the scope of the disclosure.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, formulation and medical treatments in cardiology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); Textbook of Interventional Cardiology, 7th Edition, Authors: Eric J. Topol & Paul S. Teirstein; and Animal Cell Culture: Practical Approach, Third Edition (John R. W. Masters, ed., 2000), ISBN 0199637970, whole of text; each of these references are incorporated herein by reference in their entireties.

Extracellular vesicles (EV) are lipid bound vesicles that are excreted by almost all cells into the extracellular space. EVs vary in size and exosomes are a type of EV enclosed in a single outer membrane that range, in size, between 30 to 150 nm in diameter (Doyle 2019 Cell). Exosomes are found in all body fluids including blood, cerebrospinal fluid, urine, lymph, bile and breast milk. EVs, in general, and exosomes, in particular, contain a cargo of nucleic acids, proteins and lipids that are derived from the parent cell. EVs have been shown to transfer their cargo from one cell to another, mediate cell to cell communication and affect the biology of recipient cells in a wide variety of settings. In tissue culture systems, EVs can play a role in antigen-presentation and immune and inflammation regulation. EVs are postulated to play protean roles in cancer biology. In both in vitro and in vivo models, they play a role in cell cycle and proliferation, angiogenesis, epithelial-mesenchymal transition (EMT) and metastatic niche promotion (Kalluri Science 2020). In this patent application, we demonstrate that exosomes derived from human cerebral endothelial cells exposed to stress demonstrate potent anticancer activity across a wide variety of cancers alone or in combination with miR-214.

The present disclosure provides methods for the treatment and prevention of solid tumors and carcinomas, for example, that involves administering a therapeutically effective amount of a combination comprising mammalian stressed exosomes (which include extracellular vesicles) and a treatment selected from: a chemotherapeutic agent, a radioactive agent used in the treatment of cancer, and a cancer resection surgical procedure, to a subject in need thereof.

As used herein, the term “treat” or “treating” or “treatment” refers to clinical intervention designed to alter the natural course or outcome of a pathological condition affecting an individual undergoing said treatment. Desirable effects of treatment include decreasing the rate of progression, ameliorating or palliating the pathological state, and remission or improved prognosis of a particular disease, disorder, or condition. For example, an individual is successfully “treated”, if one or more symptoms associated with a particular disease, disorder, or condition are diminished, mitigated or eliminated. Furthermore, the terms “to treat” or “treatment” according to this disclosure include the treatment of symptoms of cerebrovascular injury, disorder or disease, the prevention or the prophylaxis of the symptoms of hyperplasia, uncontrolled cell growth, cell dysregulation, for example.

“Prevent” refers to delaying or forestalling the onset or development of a disease, development of one or more symptoms associated with such disease, disorder, or condition for a period of time from minutes to indefinitely. Prevent also means reducing risk of developing a disease, disorder, or condition. “Prevent” or “preventing” or “prevention” shall be taken to mean administering an amount of mammalian OGD extracellular vesicles, or cargo constituents from extracellular vesicles, or soluble factors derived therefrom, along with a chemotherapeutic agent and/or a radiation therapy, and/or a surgical resection procedure, to effectuate the stopping or hindering or delaying of the development or progression of a disease, disorder or condition, and/or the corresponding symptoms e.g. a liver cancer or a pancreatic cancer. “Prevent” or “preventing” or “prevention” refers to prevention or delay of the onset of the disease, disorder or condition, and/or a decrease in the level of discomfort, general malaise, or persistence of the symptoms of a given disease, disorder, or condition, in a subject relative to the symptoms that would develop and/or persist in the absence of the methods of the invention. The prevention can be complete, e.g., the total absence of disease, disorder, or conditions, and/or its corresponding symptoms. The prevention can also be partial, such that the occurrence of the disorder or disease symptoms in a subject is less than that which would have occurred without the present method.

As used herein, the term “effective amount” or “therapeutically effective amount” means the amount of mammalian OGD extracellular vesicles or stressed exosomes (i.e., exosomes extracted from stressed mammalian cells), a chemotherapeutic agent, and/or a combination thereof, sufficient to effectuate a desired physiological outcome in an individual in need of the foregoing items. The effective amount can vary among individuals depending on the health and physical condition of the individual to be treated, the taxonomic group of the individuals to be treated, the formulation of the composition, assessment of the individual's medical condition, and other relevant factors. Moreover, as used herein, the term “therapeutically effective amount” refers to the minimum concentration required to effect a measurable improvement of a particular disease, disorder, or condition, for example, symptoms, and comorbidity associated with a liver cancer or a pancreatic cancer. Accordingly, the therapeutically effective amount may vary based on factors such as the disease state (e.g., size, composition, and type of liver or pancreatic cancer, along with any specific biomarker signature for the cancer), age, sex, and/or weight of the patient, along with the ability of the mammalian stressed exosomes to act in concert with a chemotherapeutic agent to elicit a desired response in the individual. A therapeutically effective amount is also one in which any toxic or detrimental effects of the chemotherapeutic agent are outweighed by the therapeutically beneficial effects.

A “suboptimal amount” is an amount that is below the optimal or standard minimum concentration required to effect a measurable improvement of a particular disease, disorder, or condition. In some embodiments, the chemotherapeutic agent by itself (i.e., when not used in combination with mammalian exosomes) may be a suboptimal amount; however, in some embodiments, when a suboptimal amount of a chemotherapeutic agent is used with mammalian exosomes, the suboptimal amount of chemotherapeutic agent may be a therapeutically effective amount.

As used herein, the term “therapeutically effective combination” or “therapeutically effective amount of a combination” (used synonymously) refers to the result or product of combining two or more agents, elements, drugs, and/or treatments (e.g., mammalian stressed extracellular exosomes and a chemotherapeutic agent, or mammalian OGD extracellular vesicles, or radiation therapy, and/or surgical resection), the combination of which results in at least the minimum combined concentration required to effect a measurable improvement of a particular disease, disorder, or condition, carcinoma oncogenesis, neoplastic growth and tumor formation. The therapeutically effective combination may vary based on factors such as the disease state (e.g., size, composition, and age of the carcinoma; specific tissues involved), age, sex, and/or weight of the patient, along ability of the mammalian exosomes in concert with a chemotherapeutic and/or surgical resection to elicit a desired response in the individual. A therapeutically effective combination is also one in which any toxic or detrimental effects of the mammalian exosomes are outweighed by the therapeutically beneficial effects.

“About” means within plus or minus (+) 10% of a value. For example, if it is stated, “a marker may be increased by about 50%”, it is implied that the marker may be increased between 45%-55%, inclusive of the endpoints and all integers or fractions thereof between the stated ranges.

“Amelioration” refers to a lessening of at least one indicator, sign, or symptom of an associated disease, disorder, or condition. The severity of indicators can be determined by subjective or objective measures, which are known to those skilled in the art.

As used herein, the terms “cancer” and “cancerous” refer to or describe the physiological condition in mammals that is typically characterized by unregulated cell growth, i.e., proliferative disorders. Examples of such proliferative disorders include cancers such as carcinoma, lymphoma, blastoma, sarcoma, and leukemia, as well as other cancers disclosed herein. More particular examples of such cancers include breast cancer, prostate cancer, colon cancer, squamous cell cancer, small-cell lung cancer, non-small cell lung cancer, gastrointestinal cancer, pancreatic cancer, cervical cancer, pancreatic cancer, liver cancer (e.g., hepatocellular carcinoma (HCC), hepatoblastoma, and cholangiocarcinoma), bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); pancreatic cancer; retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Parenteral administration” means administration by a manner other than through the digestive tract. Parenteral administration includes topical administration, subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Patient” or “Subject” are used interchangeably and for the purposes of the present disclosure includes humans and other animals, particularly mammals, and other organisms. Thus, the methods are applicable to both human therapy and veterinary applications. More specifically, the patient is a mammal, and in some embodiments, the patient or subject is human.

“Pharmaceutical composition” means a mixture of substances suitable for administering to an individual. For example, a pharmaceutical composition can comprise one or more active agents and a sterile aqueous solution.

“Pharmaceutically acceptable carrier” means a medium or diluent that does not interfere with the structure or function of the oligonucleotide. Certain, of such carries enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection or infusion. For example, a pharmaceutically acceptable carrier can be a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Pharmaceutically effective amount” for purposes herein is thus determined by such considerations as are known in the art, and may also include “therapeutically effective amounts” (also used synonymously) which is broadly used herein to mean an amount of mammalian exosomes, and chemotherapeutic agent, that when administered to a patient, ameliorates, diminishes, improves or prevents a symptom of cancer in a patient who has liver or pancreatic cancer. The amount of mammalian exosomes or their internal components, and/or the amount of chemotherapeutic agent, described herein, which constitutes a “therapeutically effective amount” where applicable, will vary depending on the agent density, the disease state and its severity, the age of the patient to be treated, and the like.

“Prophylactically effective amount” or “prophylactic amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the term “stressed cultured cells” or “stressed cultured endothelial cells” generally refers to cultured cells, which are typically mammalian cultured cells, for example, cultured mammalian endothelial cells, that are subjected to at least one of hypoxia, glucose deprivation, oxygen and glucose deprivation and combinations of these stressors. The term “hypoxic condition” or “hypoxia” refers to culturing cells or medium under conditions that are oxygen depleted compared to normal physiological oxygen levels of about 11%.

As used herein, the term “stressed mammalian exosomes” refers to exosomes and/or small extracellular vesicles (e.g., microvesicles) that include exosomes, released from cultured cells that have been exposed to conditions such as hypoxia or oxygen and glucose deprivation (OGD). Typically, it is the cells that the stressed mammalian exosomes are derived from that are stressed, which may or may not cause stress in the exosomes/microvesicles themselves. Hypoxia and oxygen deprivation refers to the culturing of the cells or medium under conditions that are oxygen depleted compared to normal physiological oxygen levels of about 11%. Glucose deprivation generally refers to the culturing of cells or medium that has glucose depleted compared to normal physiological glucose levels of about 90-150 mg/dl. The deprivation may occur for a particular time period depending on the desired implementation (e.g., at least one hour, 4 hours, 8 hours, 16 hours, 24 hours, to cite a few examples). These hypoxia and stressed exosomes have been shown to carry proteins and nucleic acids including microRNAs that are compositionally different from exosomes from the same parental sources that have not been stressed. When mammalian cells, such as mammalian endothelial cells described in the present disclosure are cultured under hypoxia or hypoxic conditions, or they are cultured under oxygen and glucose deprivation, the exosomes and microvesicles produced by these stressed mammalian cells produce “stressed mammalian exosomes”, which may include or be a part of “stressed mammalian microvesicles” or “stressed mammalian extracellular vesicles”. In some embodiments, the “exosomes” include extracellular vesicles which are themselves exosomes (size 30-150 nm), other extracellular vesicles and/or microvesicles (also known as ectosomes, shedding vesicles, microparticles, plasma membrane-derived vesicles, and exovesicles, size <1000 nm), and/or apoptotic bodies (size 1-4 μm) (D. Ha, et al. “Exosomes as therapeutic drug carriers and delivery vehicles across biological membranes: current perspectives and future challenges” (2016) Acta Pharmaceutica Sinica B, Vol 6, Issue 4, p.287-296), the disclosure of which is incorporated herein by reference in its entirety. Accordingly, “stressed mammalian extracellular vesicles” can include “stressed mammalian exosomes”. Additionally, while the disclosure herein focuses on “stressed mammalian exosomes” as a therapeutic example of a “stressed mammalian extracellular vesicle”, there may be other extracellular vesicles besides exosomes that have a therapeutic effect. However, in an advantageous embodiment, stressed mammalian exosomes were tested and used as the extracellular vesicles to help treat and prevent cancer.

Stressed cells produce a set of exosomes and microvesicles that have the capacity to inhibit the growth and destroy cancer cells in vitro and in vivo, wherein the term “stressed cells” refers to both cells cultured under hypoxia or hypoxic conditions and/or under cell culture conditions with oxygen and glucose deprivation and these conditions are used interchangeably as “stressed conditions” herein in the present disclosure to refer to hypoxic cell derived extracellular vesicles comprising exosomes and microvesicles and OGD derived extracellular vesicles comprising exosomes and microvesicles.

As used herein the term “derived from” shall be taken to indicate that a specified biological product, component or active agent may be obtained from a particular source albeit not necessarily directly from that source. For example, in the context of extracellular vesicles “derived” from a stressed mammalian cell, this term refers to stressed mammalian exosomes and/or microvesicles collectively referred to herein as “stressed exosomes” or “stressed mammalian exosomes,” that are produced by extracellular vesicle producing stressed mammalian cells, for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like, glial cells, astrocytes, neurons, oligodendrocytes, spindle neurons, microglia, or mastocytes, or in vitro cell cultures of any of the foregoing cells. In the foregoing examples, the exemplary stressed mammalian extracellular exosomes can be isolated from these exemplified cells, or may be cultured from mammalian tissue, for example, mammalian tissue or mammalian cultured cells under conditions of hypoxia and/or oxygen and glucose deprivation.

Exosomes Derived from Stressed Cells Useful in the Treatment of Cancer

In various embodiments, methods provided herein for the treatment and/or prevention of a cancer, for example, a hematological cancer or a solid tumor, include administering a therapeutically effective dose comprising stressed mammalian exosomes, which have been isolated from stressed endothelial cells, for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells that have been treated by hypoxia and/or oxygen and glucose deprivation (OGD) for at least one hour (resulting in the formation of stressed mammalian cells). In further embodiments, stressed exosomes isolated from stressed mammalian cells can be combined with one or more chemotherapeutic agents, for use concomitantly or sequentially in any regimen, useful for the treatment of liver and/or pancreatic cancer, to the subject in need thereof. In some embodiments, the stressed mammalian exosomes can be administered without the addition of any further excipient, carrier or diluent, or in the form of a composition containing the stressed mammalian exosomes admixed with one or more excipients, carriers or diluents. In various embodiments, the compositions may include non-pharmaceutical compositions or pharmaceutical compositions approved for administration to a subject, for example a human subject.

In some embodiments, illustrative stressed mammalian exosomes may include exosomes which contain within, among other proteins (e.g. Alix and/or CD63 and/or CD133), growth factors, microRNAs (miRs), siRNAs and mRNAs. In various embodiments, illustrative stressed exosomes may include their extracellular vesicles and/or stressed mammalian microvesicles that will also contain microRNA-214 (miR-214). In some embodiments, the stressed exosomes are non-enriched, in that they are not specifically transformed recombinantly (non-naturally) with an exogenous nucleic acid, for example and nucleic acid, which includes a microRNA, for example, miR-214. In some embodiments, illustrative stressed mammalian exosomes may also include stressed mammalian cell derived extracellular vesicles that may contain little to no miR-214, but which are transformed with a polynucleotide operable to express miR-214 coding nucleic acids, for example, plasmids which contain polynucleotides operable to encode miR-214 in the target cell. These stressed exosomes are said to be enriched with this miR.

In some embodiments, mammalian cells which are operable to produce exosomes of the present invention may include mammalian cells such as cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells. In some embodiments, these mammalian cells are optionally manipulated by introducing one or more miRs encoding nucleic acids for example, miR-214 which produce exosomes and/or microvesicles that contain or are capable of expressing miR-214, a vesicle containing miR-214, or a particle containing miR-214, or agents which induce the expression of miR-214 in a target cell, or in the target tissue. These mammalian cells, harboring miR-214 or not, are then cultured in vitro under conditions that deprive these mammalian cells from oxygen and/or glucose for a period of time ranging from 30 minutes to 24 hours, preferably from about 1 hour to about 12 hours and most preferably, from about one hour to about 8 hours, to generate stressed mammalian cells that are capable of producing stressed exosomes, that optionally contain miR-214. These stressed exosomes from these stressed mammalian cells contain therapeutic proteins, nucleic acids (including miRs) and lipids, that in combination are effective in treating liver cancers described herein, and pancreatic cancers as described herein, in a subject with said cancer. In various embodiments of the present disclosure, the methods of treatment and/or prevention of a liver cancer or a pancreatic cancer may include administering a therapeutically effective dose comprising a combination of OGD mammalian cell exosomes and optionally in combination with a chemotherapeutic agent to the subject in need thereof. As used herein “mammalian cell exosome cargo” refers to the internal constituents of the above referenced OGD mammalian cell exosomes, which may include a variety of proteins (e.g. Alix, CD63, CD133 or Tsg101), growth factors, miRs, siRNAs and mRNAs, for example, miR-214. In some embodiments, OGD mammalian cell exosome cargo includes internal constituents of extracellular vesicles that include miR-214 among other proteins, and nucleic acids. Accordingly, as used herein, stressed mammalian exosomes may include the extracellular vesicles (e.g., microvesicles) themselves and the mammalian cell exosome cargo. In some embodiments, the stressed mammalian exosomes may be autologous or allogeneic.

In some embodiments, endothelial cells (for example, cerebral endothelial cells (CEC), brain microvascular endothelial cells (BMVEC), Primary Human Brain Microvascular Endothelial Cells (PHBMVEC) (for example, ACBRs, antigen-presenting cells, glial cells, astrocytes, neurons, oligodendrocytes, spindle neurons, microglia, or mastocyte cells)) may be transfected with purified miR-214 based on the nucleotide sequence of miRNA-214 as shown in SEQ ID NO: 1. In an exemplary method, mammalian cells that may or may not naturally produce miRNA-214 can be transfected or transformed to produce miRNA-214, either constitutively or induced by adding an agent to a cell culture to induce production of miR-214. For example, human miR-214 may be synthesized using the nucleotide sequence 5′-UACAGCAGGCACAGACAGGCAGU-3′ (SEQ ID NO: 1). Cerebral Endothelial Cells (CECs) may be transfected and assayed using quantitative real-time polymerase chain reaction (qRT-PCR). CECs may be cultured and transfected with miR-214 according to the manufacturer's instructions using the siPORT NeoFX Transfection Agent (Applied Biosystems Inc.). Briefly, CECs may be grown in DMEM with 10% Fetal Bovine Serum (CellGro) to 80% confluence at 37° C. and 5% CO₂. Adherent cells are washed and trypsinized. Trypsin can be inactivated by re-suspending the cells in DMEM with 10% FBS (Invitrogen). The SiPORT NeoFX transfection agent is diluted in Opti-MEM I medium (Life Technologies) and incubated for 10 minutes at room temperature. miR-214 can be diluted into 50 μL Opti-MEM I medium at a concentration of 30 nM. Diluted miR-214 and diluted siPORT NeoFX Transfection Agent is mixed and incubated for another 10 minutes at room temperature to allow transfection complexes to form and subsequently dispensed into wells of a clean 6-well culture plate. The CEC suspension is overlaid onto the transfection complexes and gently mixed to equilibrate. Transfected cells are incubated at 37° C. and 5% CO₂ for 24 hours. Cells other than CECs, for example, brain microvascular endothelial cells (BMVEC), Primary Human Brain Microvascular Endothelial Cells (PHBMVEC) (for example, ACBRs, antigen-presenting cells, glial cells, astrocytes, neurons, oligodendrocytes, spindle neurons, microglia, or mastocytes), may be used and transfected with a vector which is operable to express a microRNA for example, miRNA-214 microRNA that may be packaged into an exosome and/or microvesicle for use in the oxygen and/or glucose deprivation procedure.

In some embodiments, stressed mammalian exosomes can include miR-214 microRNA. In some embodiments, stressed mammalian exosomes are devoid of miR-214, or contain less than 1% of miR-214 as a fraction of the total microRNA present in the exosome or extracellular vesicle. In some of these embodiments, methods for isolating miR-214 are known in the art. In one example, miR-214 can be produced using general, known molecular biology techniques taking advantage of the nucleotide sequence of miR-214 as shown in SEQ ID NO: 1. For example, a cDNA molecule encoding the complementary sequence of miR-214 can be cloned into a plasmid and serve as a template for polymerase chain reactions (PCR) for the synthesis of miR-214 which can then be reverse transcribed to RNA. Other methods for isolating miRNA from biological fluids are also known, for example, Lekchnov, E. A., Anal Biochem. (2016), “Protocol for miRNA isolation from biofluids”, 499:78-84. Alternatively, miR-214 can be synthesized from the nucleotide sequence of miR-214 as provided in SEQ ID NO: 1.

In other embodiments, stressed mammalian exosomes may also include natural and synthetic nucleic acid vectors (for example, plasmids, cosmids, YACs, and viral vectors) that when expressed in a mammalian cell will induce the expression of miR-214 nucleic acid sequence (for example, in the case of miR-214, a polynucleotide containing the nucleotide sequence of SEQ ID NO: 1) and which also contain expression sequences such as promoters, termination signals and other transcription and translation signals operable to express the miR-214 in its intended cells and tissues.

In some embodiments, stressed mammalian exosomes can contain miR-214. For example, mammalian cells such as: endothelial cells, (for example, cerebral endothelial cells), epithelial cells, Schwann cells, hematopoietic cells, reticulocytes, monocyte-derived dendritic cells (MDDCs), glial cells, astrocytes, neurons, oligodendrocytes, spindle neurons, microglia, or mastocytes which produce exosomes and secrete extracellular vesicles, may possess exosomes with a mammalian cell exosome cargo that contains miRNA-214 microRNA, either alone or with other mammalian exosome cargo constituents.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding a miR-19a, miR-21, or miR-146a, a vector may encode a targeting molecule. A targeting molecule is one that directs the desired nucleic acid to a particular organ, tissue, cell, or other location in a subject's body.

The term “expression vector” refers to a vector containing a nucleic acid sequence coding for at least part of a gene product capable of being transcribed. Expression vectors can contain a variety of “control sequences,” which refer to nucleic acid sequences necessary for the transcription of an operably linked coding sequence in a particular host organism. In addition to control sequences that govern transcription and translation, vectors and expression vectors may contain nucleic acid sequences that serve other functions as well and are described. There are a number of ways in which expression vectors may be introduced into cells. In certain embodiments, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. They can accommodate up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present disclosure. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Other suitable methods for nucleic acid delivery to effect expression of compositions of the present disclosure are believed to include virtually any method by which a nucleic acid (e.g., RNA, or DNA, including viral and non-viral vectors) can be introduced into an organelle, a cell, a tissue or an organism, as described herein or as would be known to one of ordinary skill in the art. Such methods include, but are not limited to, direct delivery of RNA such as by injection (U.S. Pat. Nos. 5,994,624; 5,981,274; 5,945,100; 5,780,448; 5,736,524; 5,702,932; 5,656,610; 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783; 5,563,055; 5,550,318; 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

For example, mammalian cells can be modified to engineer expression of miR-214. Additionally, in some illustrative embodiments, mammalian cells can be transfected or transformed with nucleic acid vectors, introducing nucleic acids encoding miR-214. An illustrative example of miR-214 transfection includes, but is not limited to, obtaining pre-miR-214; plating cells on a suitable cell culture dish at 50% confluence; transfecting the pre-miRNA using Lipofectamine (or any other suitable transfection agent); confirming transfection using quantitative-PCR; washing the cells twice with PBS; and extracting the miR-214 using conventional, commercially available techniques, such as the mirVana miRNA isolation kit with phenol (Thermo Fisher Scientific) (Hu et al., MicroRNAs 125a and 455 Repress Lipoprotein-Supported Steroidogenesis by Targeting Scavenger Receptor Class B Type I in Steroidogenic Cells, Mol Cell Biol. 2012 December; 32 (24): 5035-5045, the disclosure of which is incorporated herein by reference in its entirety).

In some embodiments, mammalian cells operable to produce and secrete exosomes can be transfected with miR-214 using common techniques known to those with ordinary skill in the art, and/or by using commercially available kits (e.g., Exo-fect Exosome Transfection Kit, System Biosciences). Furthermore, cells can be reprogrammed to express mammalian exosomes and/or miR-214. An exemplary miRNA reprogramming method is illustrated by Trivedi et al., “Modification of tumor cell exosome content by transfection with wt-p53 and microRNA-125b expressing plasmid DNA and its effect on macrophage polarization”, Oncogenesis. 2016 August; 5 (8): e250, the disclosure of which is incorporated herein by reference in its entirety. In a non-limiting embodiment, a plasmid containing pre-miR-19a, pre-miR-21, or pre-miR-146a microRNA is isolated and purified. Next, hyaluronic acid-poly(ethylene imine) and hyaluronic acid (HA)-poly(ethylene glycol) (PEG) (HA-PEI/HA-PEG) blend nanoparticles are then obtained by combining 50 mg of maleimide-PEG-amine to 1-Ethyl-3-(3-dimethylaminopropyl)-carbodimide (EDC)/N-hydroxysuccinimide (NHS) activated HA, and dissolving the HA-PEI and HA-PEG solutions in PBS. Cells such as stem cells, mesenchymal stromal cells, umbilical cord cells, endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like)), epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, or in vitro cell cultures of any of the foregoing cells, or any cell with an endomembrane system, can be plated and treated with a suitable amount of plasmid containing miR-214 (e.g., 1-20 μg) encapsulated in the nanoparticles. Finally, exosomes can be isolated using techniques described above, by using commercially available kits, or by taking cell supernatant from, and centrifuging at 2000 g for 30 min to remove cell debris; taking the supernatant and adding it to a commercially available exosome isolation reagent, followed by incubation overnight at 4° C.; further centrifuged at 10,000 g for 1 hour at 4° C.; and aspiration of the supernatant followed by resuspending the exosome pellet in sterile PBS.

In some embodiments, cells can be induced to release and/or secrete extracellular vesicles in response to a variety of signals including, but not limited to, cytokines, mitogens, and/or any other method of paracrine/autocrine signaling (see Saunderson et al., “Induction of Exosome Release in Primary B Cells Stimulated via CD40 and the IL-4 Receptor”, J Immunol. 2008 Jun. 15; 180 (12): 8146-52, the disclosure of which is incorporated herein by reference in its entirety).

In some embodiments, mammalian cells can be induced to release and/or secrete exosomes by modulating intracellular calcium (Ca²⁺) content. An exemplary illustrative technique for stimulating a mammalian exosome and/or a microvesicle containing miR-214 is provided by Savina et al., “Exosome release is regulated by a calcium-dependent mechanism in K562 cells”, the disclosure of which is incorporated herein by reference in its entirety. After selecting the suitable cell type, for example, stem cells, mesenchymal stromal cells, umbilical cord cells, endothelial cells, (for example, cerebral endothelial cells (CEC), brain microvascular endothelial cells (BMVEC), Primary Human Brain Microvascular Endothelial Cells (PHBMVEC) (for example, ACBRs, antigen-presenting cells, glial cells, astrocytes, neurons, oligodendrocytes, spindle neurons, microglia, or mastocytes and/or any cell with an endomembrane system, a compound that influences Na⁺/H⁺ exchange and/or intracellular calcium (Ca²⁺) content (e.g., an ionophore such a monesin)), can be applied to stimulate mammalian exosome release. Subsequent to mammalian exosome stimulation, the exosomes can be isolated using any one of the techniques known to those with ordinary skill, and/or enumerated herein.

Some embodiments may call for stressed mammalian exosomes, for example, exosomes to be produced by stimulating and/or inducing the overproduction of extracellular vesicles in either stressed endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells, or any one or more of the abovementioned cells, and/or any cell with an endomembrane system, that has been transformed or transfected to overexpress miR-214), using techniques known to those with ordinary skill, and/or enumerated herein, for example as provided in: Amigorena S, Raposo G, Clayton A: “Isolation and characterization of exosomes from cell culture supernatants and biological fluids”. Curr. Protoc. Cell Biol. 2006 April; Chapter 3: Unit 3.22, the disclosure of which is incorporated herein by reference in its entirety. Typically, 100 mL of cultured media is used by pooling from multiple dishes. The media is centrifuged at 300×g for 10 min at 4° C. to remove any intact cells, followed by a 2,000×g spin for 20 min at 4° C. to remove dead cells and finally a 10,000×g spin for 30 min at 4° C. to remove cell debris. The media is then transferred to ultracentrifuge tubes and centrifuged at 100,000×g for at least 60 min at 4° C. in Optima TLX ultracentrifuge with 60 Ti rotor (Beckman Coulter, Mississauga, Canada). The supernatant containing exosome-free media is removed and the pellets containing exosomes plus proteins from media are resuspended in PBS. The suspension is centrifuged at 100,000×g for at least 60 min at 4° C. to collect final exosome pellets. The exosome pellet is then resuspended in an appropriate excipient or diluent in a desired volume to attain a specific concentration of exosomes per mL.

Exosomes may also be isolated using any of the techniques described by Willis et al., Toward Exosome-Based Therapeutics: Isolation, Heterogeneity, and Fit-for-Purpose Potency (2017) Front Cardiovasc Med. 4:63, the disclosure of which is incorporated herein by reference in its entirety. Such isolation methods include Ultracentrifugation (i.e., 100,000-120,000×g); size-exclusion chromatography; commercially available isolation kits (e.g. ExoQuick and ExoELISA); and CD63 capture (exosome) ELISA, (Systems Biosciences, CA, USA).

An exemplary exosomes isolation method can be adapted from R. Szatanek et al. Isolation of extracellular vesicles: Determining the correct approach (2015) Int J Mol Med. 2015 July; 36 (1): 11-17, the disclosure of which is incorporated herein by reference in its entirety. Typically, for differential centrifugation/ultracentrifugation, intact cells, dead cells and cell debris are removed by centrifuging at 300×g for 10 min, 2,000×g for 10 min and 10,000×g for 30 min, respectively. Supernatant is transferred into a new test tube while the generated pellets are being discarded. After the 10,000×g spin, the supernatant is then subjected to a final ultracentrifugation at 100,000×g for 70 min, all centrifugation steps carried out at 4° C.

In some embodiments, the methods described herein can utilize compositions and/or formulations containing stressed exosomes derived from a variety of exosome producing mammalian cells, for example, endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells which have been stressed, i.e. deprived of oxygen and/or glucose for at least one hour in cell culture.

In various embodiments, mammalian cell derived stressed exosomes include exosomes from mammalian cells which are operable to produce and secrete exosomes containing miR-214. In some embodiments, compositions of the present disclosure comprise stressed exosomes derived from stressed cerebral endothelial cells (CECs). In closely related embodiments, compositions containing stressed exosomes include compositions containing stressed CEC derived exosomes in which at least a portion of the stressed exosomes optionally contain miR-214. In some embodiments, compositions of the present disclosure comprise stressed exosomes derived from endothelial cell progenitor cells (for example, AG-133/CD-133+ cells and the like). In closely related embodiments, compositions containing stressed exosomes include compositions containing endothelial cell progenitor cell derived stressed exosomes in which optionally, at least a portion of stressed exosomes contain miR-214.

An exemplary CEC isolation method can be adapted from Ruck et al., Isolation of Primary Murine Brain Microvascular Endothelial Cells, J Vis Exp. 2014; (93): 52204, the disclosure of which is incorporated herein by reference in its entirety. Alternatively, CECs may be obtained using the commercially available Microvascular Endothelial Cell Growth Kit-BBE (ATCC® PCS-110-040TM), or the PrimaCell™, Rat Cerebral Venous Vascular Endothelial Cell Culture Kit (CHI Scientific).

In some embodiments, the compositions of the present disclosure may also comprise a chemotherapeutic agent, either in admixture with the stressed exosomes, or in a separate composition for administration to a patient having a liver cancer or an pancreatic cancer, in need thereof. The chemotherapeutic agent can include one or more of alkylating agents, anti-metabolites, antitumor antibiotics, antimitotic agents, topoisomerase I and II inhibitors, hormones and hormonal analogues, retinoids, signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the compounds of the invention in the compositions and methods described above. Examples of alkylating agents include but are not limited to nitrogen mustards such as cyclophosphamides, temozolamide, melphalan, and chlorambucil; oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the compounds of the invention in the compositions and methods described above. Examples of antimetabolite anti-neoplastic agents include but are not limited to purine and pyrimidine analogues and anti-folate compounds, and more specifically, hydroxyurea, cytosine, arabinoside, ralitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the compounds of the invention in the compositions and methods described above.

Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the compounds of the invention in the compositions and methods described above. Examples of antimitotic agents include, but are not limited to, diterpenoids, vinca alkaloids, polo-like kinase (Plk) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine. Plk inhibitors are discussed further below.

Topoisomerase inhibitors include inhibitors of Topoisomerase II and inhibitors of Topoisomerase I. Topoisomerase II inhibitors, such as epipodophyllotoxins, are anti-neoplastic agents derived from the mandrake plant, e.g., Podophyllum sp., that typically affect cells in the S and G2 phases of the cell cycle by forming a ternary complex with topoisomerase II and DNA, causing DNA strand breaks. The strand breaks accumulate and cell death follows. Examples of epipodophyllotoxins include, but are not limited to, etoposide and teniposide. Camptothecins, including camptothecin and camptothecin derivatives, are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the CEC exosomes of the invention in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the compounds of the invention in the compositions and methods described above. Examples of hormones and hormonal analogues believed to be useful in the treatment of neoplasms include, but are not limited to antiestrogens, such as tamoxifen, toremifene, raloxifene, fulvestrant, iodoxyfene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrazole, vorazole, and exemestane; progestrins such as megestrol acetate; 5α-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

Retinoid(s) are compounds that bind to and activate at least one retinoic acid receptor selected from RARα, RARβ, and RARγ and/or compounds that bind to and activate at least one of RARα, RARβ, and RARγ and also at least one retinoic X receptor (RXR), including RXRα, RXRβ, and RXRγ. Retinoids for use in the present invention typically have affinity for RAR, and particularly for RARα and/or RARβ. However, certain synthetic retinoids, such as 9-cis-retinoic acid also have affinity for both RAR and RXR. In one embodiment, the retinoid has affinity for RARα (and RARα agonist).

Signal transduction pathway inhibitors are those inhibitors which block or inhibit a chemical process which evokes an intracellular change. As used herein these changes include, but are not limited to, cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3—OH kinases, myoinositol signaling, and Ras oncogenes. Signal transduction pathway inhibitors may be employed in combination with the compounds of the invention in the compositions and methods described above.

Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1R, IR and IRR); epidermal growth factor family receptors (EGFR, ErbB2, and ErbB4); platelet derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET protooncogene.

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb™) and erlotinib (TARCEVA®). Imatinib (GLEEVEC®) is one example of a PDGFR inhibitor. Examples of VEGFR inhibitors include pazopanib, ZD6474, AZD2171, PTK787, sunitinib and sorafenib.

In some embodiments, compositions comprising a chemotherapeutic agent may contain the chemotherapeutic agent at concentrations ranging from about 100 mg/mL to about 0.001 mg/mL, or from about 100 mg/mL to about 0.01 mg/mL, or any range therebetween. In various embodiments, the chemotherapeutic agent is provided in a composition at concentrations that have been approved for use in the treatment of liver cancer or an pancreatic cancer in the United States or in Europe.

In other embodiments, illustrative stressed mammalian exosomes can include exosomes derived from a mammalian cell (for example, a human cell) that synthesizes and expresses miR-214, and packages same into an exosome and/or microvesicle. In some embodiments, stressed mammalian cells can be administered to treat and prevent a liver cancer or a pancreatic cancer or the symptoms of a liver cancer or a pancreatic cancer by administering a population of stressed mammalian cells that naturally produce and secrete stressed exosomes (accordingly, treatment with the stressed cells and/or stressed extracellular vesicles may constitute treatment using stressed exosomes). In some of these embodiments, the stressed exosomes can be characterized in that the exosomes or extracellular vesicles express a protein selected from Alix, CD63, CD133 or Tsg101.

In some of these embodiments, these stressed exosomes may also contain one or more miRNAs including miR-214, for example, stressed mammalian cells (for example, human): endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes which produce at least one protein selected from Alix, CD63, CD133 or Tsg101, and/or miR-214.

As used herein, the term “chemotherapeutic agent” or “anti-neoplastic agent” or anti-cancer agent” (used synonymously herein) refers to agents selected from: alkylating agents, anti-metabolites, antitumor antibiotics, antimitotic agents, topoisomerase I and II inhibitors, hormones and hormonal analogues; retinoids, signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

In some embodiments, without limitation, the methods described herein may utilize compositions and/or formulations containing stressed mammalian cell-derived exosomes. In further related embodiments, methods described herein may utilize compositions and/or formulations containing stressed mammalian cell-derived exosomes in combination with a chemotherapeutic agent. In various embodiments, the compositions of the present methods are administered separately or may be administered concomitantly, i.e. administered on the same day, in the same hour, or administered immediately after the other. In other embodiments, an illustrative composition comprises mammalian cell derived stressed exosomes and one or more chemotherapeutic agents in a single composition. In some embodiments, the mammalian stressed exosomes useful in the methods of the present disclosure include stressed exosomes derived from microvesicles and/or stressed mammalian cells, for example, a human cell selected from: endothelial cells (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells, which are treated by depriving said mammalian cells from oxygen and/or glucose for at least one hour, to produce stressed exosomes and secrete stressed extracellular vesicles. In various embodiments, stressed mammalian exosomes are derived or isolated from stressed endothelial cell progenitor cells (for example, AG-133/CD-133+ cells) and/or cerebral endothelial cells (CECs) that have been stressed in culture, i.e. cultured under hypoxia and/or deprived of oxygen and glucose for at least one hour. The foregoing stressed mammalian cells can be obtained via primary cell culture, or through commercial vendors. For example, cerebral endothelial cells are commercially available from the American Type Culture Collection (ATCC), Manassas, VA, USA, and may be grown and processed in the methods described herein, to produce stressed exosomes, for example, stressed exosomes.

In various embodiments, the compositions comprising mammalian derived stressed exosomes include an stressed extracellular vesicle, for example, a stressed exosome or a stressed microvesicle containing at least three of the following characteristic exosome constituents:

In some embodiments, the stressed mammalian cells can be manipulated as described above to introduce one or more microRNAs, including, without limitation, miR-214 either before the mammalian cells are deprived of oxygen and/or glucose, or after the mammalian cells have been deprived of oxygen and/or glucose. In some embodiments, compositions of the present disclosure may comprise: mammalian stressed exosomes which contain miR-214, human cells that are operable to synthesize exosomes containing miR-214, or particles containing miR-214 for example, liposomes, microparticles, nanoparticles, or other common vehicles for delivery of nucleic acid commonly known in the art.

In some embodiments, mammalian stressed exosomes can include particles derived from living cells, for example mammalian cells which are deprived of oxygen and/or glucose during the growth or processing of said living cells. In some embodiments, mammalian cells include cells that are known to produce exosomes, and microvesicles, for example, endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or in vitro cell cultures of any of the foregoing cells.

As used herein, the term “individual” or “subject” or “mammal” means a human or non-human mammal selected for treatment or therapy.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

### Compositions

Methods for preparing a formulation of mammalian exosomes are known, and/or are readily apparent to those skilled in the art. An exemplary formulation method can be adapted from Remington's Pharmaceutical Sciences (17th Ed., Mack Pub. Co. 1985); Remington: Essentials of Pharmaceutics (Pharmaceutical Press, 2012), the disclosure of which is incorporated herein by reference in its entirety. Methods for formulating a nucleic acid, for example, miR-19a, miR-21, or miR-146a microRNA and a pharmaceutically acceptable vehicle, carrier, or excipient for the delivery of nucleic acids are provided in U.S. Patent Application Publication No. US 2013/0017223A1, Ser. No. 13/516,335, filed on Dec. 17, 2016, the disclosure of which is incorporated herein by reference in its entirety. Methods for formulating a pharmaceutically acceptable vehicle, carrier, or excipient for the delivery of miRNA are provided in U.S. Patent No. U.S. Pat. No. 9,301,969B2, Ser. No. 13/822,641, filed on Sep. 9, 2011, the disclosure of which is incorporated herein by reference in its entirety. Methods for preparing a formulation of exosomes containing an agent are provided in U.S. Patent Application Publication No. US 2013/0156801A1, Ser. No. 13/327,244, filed on Dec. 15, 2011, the disclosure of which is incorporated herein by reference in its entirety. Furthermore, methods for preparing formulations for the exosome mediated delivery of biotherapeutics are provided in World Intellectual Property Organization Patent Application Publication No. WO 2013/084000 A2, filed on Dec. 7, 2012; and U.S. Patent Application Publication No. US 2016/0346334 A1, Ser. No. 15/116,579, filed on Feb. 5, 2015, the disclosures of these patent references are incorporated herein by reference in their entirety.

In some embodiments, without limitation, the methods described herein can utilize compositions and formulations containing one or more isolated mammalian stressed exosomes that are contained within a pharmaceutically acceptable vehicle, carrier, adjuvants, additives and/or excipient that allows for storage and handling of the agents before and during administration. Moreover, in accordance with certain aspects of the present disclosure, the agents suitable for administration may be provided in a pharmaceutically acceptable vehicle, carrier, or excipient with or without an inert diluent. Further, in addition to the above-described components, the formulation may contain additional lubricants, emulsifiers, suspending-agents, preservatives, or the like. Accordingly, the pharmaceutically acceptable vehicle, carrier, adjuvants, additives and/or excipient must be acceptable in the sense of being compatible with the other ingredients of the formulation and not deleterious to the recipient thereof, i.e., are sterile compositions and contain pharmaceutically acceptable vehicle, carrier, adjuvants, additives that are approved by the US Food and Drug Administration (FDA) for administration to a human subject.

Formulations containing stressed mammalian exosomes may be prepared with one or more carriers, excipients, and diluents. Exemplary carriers, excipients and diluents can include one or more of sterile saline, phosphate buffers, Ringer's solution, and/or other physiological solutions that are used in the preparation of cellular therapies for administration in humans. An exemplary method for generating formulations containing mammalian exosomes is illustrated by Haqqani et al., “Method for isolation and molecular characterization of extracellular microvesicles released from brain endothelial cells,” Fluids Barriers CNS. 2013 January 10; 10 (1): 4, the disclosures of which are incorporated herein by reference in its entirety. An alternative method for generating formulations containing mammalian exosomes is illustrated by Li et al., “Exosomes Derived from Human Umbilical Cord Mesenchymal Stem Cells Alleviate Liver Fibrosis”. Stem Cells Dev. 2013; 22:845-854, and Qiao et al., “Human mesenchymal stem cells isolated from the umbilical cord”, Cell Biol Int. 2008 January; 32 (1): 8-15. Epub 2007 Aug. 19, the disclosures of which are incorporated herein by reference in their entireties.

In certain embodiments, formulations comprising one or more mammalian stressed exosomes can contain further additives including, but not limited to, pH-adjusting additives, osmolarity adjusters, tonicity adjusters, anti-oxidants, reducing agents, and preservatives. Useful pH-adjusting agents include acids, such as hydrochloric acid, bases or buffers, such as sodium lactate, sodium acetate, sodium phosphate, sodium citrate, sodium borate, or sodium gluconate. Further, the compositions of the invention can contain microbial preservatives. Useful microbial preservatives include methylparaben, propylparaben, and benzyl alcohol. The microbial preservative is typically employed when the formulation is placed in a vial designed for multidose use. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., .alpha.-tocopherol (vitamin E)), preservatives, chelating agents (e.g., EDTA and/or EGTA), viscomodulators, tonicifiers (e.g., a sugar such as sucrose, lactose, and/or mannitol), flavorants, colorants, odorants, opacifiers, suspending agents, binders, fillers, plasticizers, lubricants, and mixtures thereof. The amounts of such additives can be readily determined by one skilled in the art, according to the particular properties desired. Further, the formulation may comprise different types of carriers suitable for liquid, solid, or aerosol delivery.

In certain embodiments, a formulation can be made by suspending mammalian stressed exosomes in a physiological buffer with physiological pH, for example, a sterile buffer solution such as phosphate buffer solution (PBS); sterile 0.85% NaCl solution in water; or 0.9% NaCl solution in Phosphate buffer having KCl. Physiological buffers (i.e., a 1×PBS buffer) can be prepared, for example, by mixing 8 g of NaCl; 0.2g of KCl; 1.44g of Na₂HPO₄; 0.24 g of KH₂PO₄; then, adjusting the pH to 7.4 with HCl; adjusting the volume to IL with additional distilled H₂O; and sterilizing by autoclaving.

In some embodiments, methods for the treatment and prevention of a liver cancer or pancreatic cancer may include administration of a formulation containing mammalian stressed exosomes to be combined with a biological fluid such as blood, nasal secretions, saliva, urine, breast milk, cerebrospinal fluid, and/or any other natural matrix that represents a minimalist processing step (i.e., a step/storage component that reduces the possibility of influencing mammalian surface exosome characteristics and/or behavior/integrity upon introduction to the subject/patient); an exemplary illustrative technique for formulating stressed mammalian exosomes, with one of the aforementioned biofluids, is provided by Witwer et al., Standardization of sample collection, isolation and analysis methods in extracellular vesicle research, J Extracell Vesicles. 2013; 2, the disclosure of which is incorporated herein by reference in its entirety”.

In some embodiments, the potency/quantity of a formulation containing mammalian stressed exosomes can be quantified using conventional tools and techniques known to those having ordinary skill in the art, e.g., the electrical resistance nano pulse method, using commercially available tools and components, to determine the yield of an exosome preparation (e.g., qNano; IZON Science Ltd., Oxford, UK) (see Komaki et al., Exosomes of human placenta-derived mesenchymal stem cells stimulate angiogenesis, Stem Cell Res Ther. 2017; 8:219, the disclosure of which is incorporated herein by reference in its entirety). Furthermore, the dosage of exosomes, and/or a composition containing the contents of the aforementioned mammalian cell derived exosomes, may also be confirmed/quantified using the tools available to one having ordinary skill such as tunable resistive pulse sensing, protein quantification (e.g., Protein Assay Rapid Kit, Wako Pure Chemicals, Osaka, Japan), nanoparticle tracking analysis, enzyme-linked immunosorbent assay (ELISA), flow cytometry, dynamic light scattering, cell equivalents, fingerprinting (i.e., quantifying surrogate markers as an indication), and/or using a sample to elicit a response on an in vitro/in vivo surrogate (see Willis et al., Toward Exosome-Based Therapeutics: Isolation, Heterogeneity, and Fit-for-Purpose Potency, Front Cardiovasc Med. 2017; 4:63, the disclosure of which is incorporated herein by reference in its entirety).

Prior to administration, in some embodiments, depending on the quantity and/or content of the mammalian stressed exosomes-will require appropriate storage and/or handling, the process and/or conditions of which should be dictated by the said quality/content of the stressed mammalian exosomes, and good medical practice. For example, in some non-limiting embodiments, a mammalian exosome formulation that includes the extracellular vesicles, for example, endothelial cell progenitor and/or CEC derived extracellular vesicles; or pharmaceutically acceptable compositions containing CEC derived extracellular vesicles described herein, with any one of the abovementioned carriers, excipients, and diluents, may be stored at −20° C., for a length of time that will not degrade the stressed mammalian exosomes. Storage formulations that have been successful include buffers that resist pH shifts during freezing/thawing, and are devoid of glycerol and/or dimethyl sulfoxide (see Willis et al., Toward Exosome-Based Therapeutics: Isolation, Heterogeneity, and Fit-for-Purpose Potency, Front Cardiovasc Med. 2017; 4:63, the disclosure of which is incorporated herein by reference in its entirety). Furthermore, in some non-limiting embodiments, the container should be tailored to the stressed mammalian exosomes, and should consist of a material that supports mammalian exosome storage (e.g., cell culture/clinical grade glassware or plastic) (see Lener et al., Applying extracellular vesicles based therapeutics in clinical trials, J Extracell Vesicles. 2015; 4:10.3402/jev.v4.30087, the disclosure of which is incorporated herein by reference in its entirety).

When necessary, proper fluidity of the compositions and formulations described herein can be maintained, for example, by the use of a coating such as lecithin, by the maintenance of the required size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for such compositions of mammalian exosomes. Furthermore, various additives which enhance the stability, sterility, and/or isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. Prolonged absorption of the injectable pharmaceutical form can be brought about by the use of agents delaying absorption, for example, aluminum monostearate and gelatin. According to some embodiments of the present disclosure, however, any vehicle, diluent, or additive used would have to be compatible with mammalian exosomes.

Sterile injectable solutions can be prepared by incorporating mammalian stressed exosomes utilized in practicing some embodiments of the present disclosure in the required amount of the appropriate solvent with various other ingredients, as desired.

In some non-limiting embodiments, a formulation can be prepared by combining mammalian stressed exosomes isolated from either stressed endothelial cells (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes. In some embodiments, the mammalian cell derived stressed exosomes optionally contain miR-214 microRNA. In some illustrative embodiments, a formulation may comprise one or more of stressed endothelial cell progenitor stressed exosomes and/or stressed CEC derived exosomes; and a pharmaceutically acceptable carrier, excipient, or diluent. In some embodiments, a formulation containing a mammalian stressed exosomes can include a composition comprising CEC derived stressed exosomes optionally containing miR-214 microRNA described herein, in addition to any one or more of the abovementioned carriers, excipients, and diluents.

Formulations containing mammalian exosomes stressed and a chemotherapeutic agent may be prepared with one or more carriers, excipients, and diluents. Exemplary carriers, excipients and diluents can include one or more of sterile saline, phosphate buffers, Ringer's solution, and/or other physiological solutions that are used in the preparation of cellular therapies for administration in humans. Alternatively, in some embodiments, the chemotherapeutic agent or agents may be formulated separately from stressed mammalian exosomes. For example, a chemotherapeutic agent may be supplied as lyophilized form, or in a concentrated form, to be resuspended and diluted to the final effective dose in 0.9% Sodium Chloride solution, or 5% Dextrose Injection solution.

### Administration

As used herein, the term “administering” means providing an agent to a subject in need thereof, and includes, but is not limited to, administering by a medical professional and self-administering. In some embodiments, without limitation, the methods described herein can be administered intravenously; intraarterially; subcutaneously; intramuscularly; intraperitoneally; stereotactically; intranasally; mucosally; intravitreally; intrastriatally; or intrathecally. The foregoing administration routes can be accomplished via implantable microbead (e.g., microspheres, sol-gel, hydrogels); injection; continuous infusion; localized perfusion; catheter; or by lavage. In some embodiments, the compositions and formulations of the present disclosure are administered via injection or infusion, preferably by intravenous, subcutaneous, or intraarterial administration. Methods for administering a formulation of a mammalian Stressed extracellular vesicles and a therapeutic agent can adapted from Remington's Pharmaceutical Sciences (17th Ed., Mack Pub. Co. 1985), the disclosure of which is incorporated herein by reference in its entirety.

In various embodiments, methods are provided for the prevention and/or treatment of a liver cancer or a pancreatic cancer, or the metastasis of same in a subject, comprising administering to the subject in need thereof, a therapeutically effective amount of stressed mammalian exosomes. In some embodiments, methods are provided for the prevention and/or treatment of a liver cancer or a pancreatic cancer, or the metastasis of same in a subject, comprising administering to the subject in need thereof, a therapeutically effective amount of stressed mammalian exosomes and a chemotherapeutic agent, a therapeutically effective amount of a combination of stressed mammalian exosomes and radiation therapy, a therapeutically effective amount of a combination of stressed mammalian exosomes and a surgical resection procedure, or a therapeutically effective amount of a combination of stressed mammalian exosomes, a chemotherapeutic agent, radiation therapy, and a surgical resection procedure, in any combination and performed in any given order as best determined by a medical professional, for example, an oncologist, or oncology surgeon. The methods contemplate administering one or more compositions that are pharmaceutically acceptable for the treatment of humans, particularly humans who have a liver cancer or a pancreatic cancer and are deemed safe and effective. In various embodiments, the administration of the stressed mammalian exosomes, a chemotherapeutic agent, and/or the performance of a surgical resection procedure can be accomplished using an administration method known to those of ordinary skill in the art.

Dosages for a particular patient can be determined by one of ordinary skill in the art using conventional considerations, (e.g., by means of an appropriate, conventional pharmacological protocol). A physician may, for example, prescribe a relatively low dose of a chemotherapeutic agent and/or mammalian stressed exosomes at first, subsequently increasing the dose until an appropriate response is obtained. A first dose of stressed exosomes, for example, stressed exosomes derived from stressed CECs can range from about 1×10⁵ to about 1×10¹⁹ stressed exosomes per dose, or per daily dose administered intravenously (IV), subcutaneously (S.C.) or orally to a subject with cancer. The dose administered to a patient is sufficient to effect a beneficial therapeutic response in the patient over time, e.g., to reduce the size of the primary tumor, and/or prevent metastasis, or other appropriate activity, for example, an objective response, a partial response, a full response, or remission of the liver cancer or pancreatic cancer, depending on the application as determined using standard measures, for example, Response Evaluation Criteria in Solid Tumors (RECIST) Guidelines. The dose is determined by the efficacy of the particular formulation, and the activity, stability or serum half-life of the mammalian exosome employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

“Dosage unit” means a form in which a pharmaceutical agent or agents are provided, e.g. a solution or other dosage unit known in the art. Further, as used herein, “Dose” means a specified quantity of a pharmaceutical agent provided in a single administration, or in a specified time period. In certain embodiments, a dose can be administered in one, two, or more, boluses, infusions, or injections. For example, in certain embodiments where intravenous or subcutaneous administration is desired, the desired dose may require a volume not easily accommodated by a single injection, therefore, two or more injections can be used to achieve the desired dose, or one or more infusions are administered. In certain embodiments, the pharmaceutical agent is administered by infusion over an extended period of time or continuously. Doses can be stated as the amount of pharmaceutical agent per hour, day, week, or month. Doses can be expressed as μg/kg, mg/kg, g/kg, mg/m² of surface area of the patient, or number of exosomes.

Therapeutic compositions comprising mammalian stressed exosomes are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the EC₅₀ of the relevant formulation, and/or observation of any side-effects of the mammalian stressed exosomes and/or chemotherapeutic agent at various concentrations, e.g., as applied to the mass and overall health of the patient. Administration can be accomplished via single or divided doses. Various factors may be used by a skilled practitioner, for example, a clinician, physician, or medical specialist to properly administer stressed mammalian exosomes, optionally in combination with a chemotherapeutic agent, and/or perform the surgical resection procedure. For example, if using a composition containing both stressed mammalian exosomes and the chemotherapeutic agent that can circulate freely in the bloodstream, the compositions or formulations of the combination may be administered intravenously, subcutaneously or intra-arterially. Similarly, separate compositions, each containing either the stressed mammalian exosomes or chemotherapeutic agent, each can be administered intravenously, subcutaneously or intra-arterially. In some embodiments, the stressed mammalian exosomes may be administered prior to, concomitantly with or subsequent to the administration of the chemotherapeutic agent. In some embodiments, the stressed mammalian exosomes are administered prior to the administration of the chemotherapeutic agent. In related embodiments, a first dose of stressed mammalian exosomes is administered as an intravenous bolus, followed by the administration of the chemotherapeutic agent, which may be administered as an infusion. The stressed mammalian exosomes and chemotherapeutic agent may be administered in various ways; for example, stressed mammalian exosomes can be administered alone, or as an active ingredient in combination with pharmaceutically acceptable carriers, diluents, adjuvants and vehicles, or in concert with one or more therapeutic agent. The stressed mammalian exosomes can be administered parenterally, for example, intravenously, intra-arterially, subcutaneously administration as well as intrathecal and infusion techniques, or by local administration or direct administration (stereotactic administration) to the site of disease or pathological condition. Repetitive administrations of the stressed mammalian exosomes, and/or chemotherapeutic agent, may also be useful, where short term or long term (for example, hours, days or weeklong administration is desirable). In various embodiments, the chemotherapeutic agent or agents may be administered parenterally, preferably by intravenous administration either by direct injection, infusion or via catheter administration as approved for the treatment of a cancer by regulatory review by a competent regulatory body, for example, the US Food and Drug Administration (FDA) or the European Medicines Agency.

The subject or patient being treated is a warm-blooded animal and, in particular, mammals, including humans. The pharmaceutically acceptable carriers, diluents, adjuvants and vehicles as well as implant carriers generally refer to inert, non-toxic solid or liquid fillers, diluents or encapsulating material not reacting with the active components of the invention. In some embodiments, mammalian exosomes may be altered by use of antibodies to cell surface proteins to specifically target tissues of interest.

“Mammal” or “mammalian” refers to a human or non-human mammal, including, but not limited to, mice, rats, rabbits, dogs, cats, pigs, and non-human primates, including, but not limited to, monkeys and chimpanzees.

In some embodiments, when administering mammalian stressed exosomes parenterally, it will generally be formulated in a unit dosage injectable form (for example, in the form of a liquid, for example, a solution, a suspension, or an emulsion). Some pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

A pharmacological formulation of some embodiments may be administered to the patient in an injectable formulation containing any compatible carrier, such as various vehicle, adjuvants, additives, and diluents; or the inhibitor(s) utilized in some embodiments may be administered parenterally to the patient in the form of slow-release subcutaneous implants or vectored delivery, iontophoretic, polymer matrices, liposomes, and microspheres. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the mammalian stressed exosomes and/or the chemotherapeutic agent. In addition, a pump-based hardware delivery system may be used to deliver one or more compositions described herein.

Examples of systems in which release occurs in bursts includes, e.g., systems in which the mammalian extracellular vesicle cargo is entrapped in liposomes which are encapsulated in a polymer matrix, the liposomes being sensitive to specific stimuli, e.g., temperature, pH, light or a degrading many other such implants, delivery systems, and modules are well known to those skilled in the art.

In some embodiments, without limitation, mammalian stressed exosomes may be administered initially by an infusion or intravenous injection. In embodiments, wherein the stressed exosomes also incorporate miR-214, the dose or doses can be administered in amounts or frequencies sufficient to bring blood levels of miR-214 to a suitable therapeutic level. The patient's levels are then maintained by an intravenous dosage form of stressed mammalian exosomes, although other forms of administration, dependent upon the patient's condition and as indicated above, can be used. The quantity to be administered and timing of administration may vary for the patient being treated.

Additionally, in some embodiments, without limitation, mammalian stressed exosomes may be administered in situ to bring internal levels to a suitable level. The patient's levels are then maintained as appropriate in accordance with good medical practice by appropriate forms of administration, dependent upon the patient's condition. The quantity to be administered and timing of administration may vary for the patient being treated.

In certain non-limiting embodiments, mammalian stressed exosomes are administered via intravenous injection, for example, a subject is injected intravenously with a formulation of mammalian exosomes suspended in a suitable carrier using a needle with a gauge ranging from about 7-gauge to 25-gauge (see Banga (2015) Therapeutic Peptides and Proteins: Formulation, Processing, and Delivery Systems; CRC Press, Boca Raton, FL). An illustrative example of intravenously mammalian exosomes includes, but is not limited to, uncovering the injection site; determining a suitable vein for injection; applying a tourniquet and waiting for the vein to swell; disinfecting the skin; pulling the skin taut in the longitudinal direction to stabilize the vein; inserting needle at an angle of about 35 degrees; puncturing the skin, and advancing the needle into the vein at a depth suitable for the subject and/or location of the vein; holding the injection means (e.g., syringe) steady; aspirating slightly; loosening the tourniquet; slowly injecting the mammalian exosomes; checking for pain, swelling, and/or hematoma; withdrawing the injection means; and applying sterile cotton wool onto the opening, and securing the cotton wool with adhesive tape.

In some embodiments, the initial administration may include an infusion of mammalian stressed exosomes via intravenous administration over a period of 1 minute to 120 minutes. Subsequent doses of the mammalian exosomes can be accomplished using intravenous injections or by infusion. Each dose administered may be therapeutically effective doses or suboptimal doses repeated if needed.

Any appropriate routes of stressed exosome and microvesicle administration known to those of ordinary skill in the art may comprise embodiments of the invention. In some embodiments, isolated mammalian stressed exosomes contained within a pharmaceutically acceptable vehicle, carrier, or excipient, or for example, exosomes derived from stressed mammalian cells, for example, endothelial cells (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or cell cultured cells thereof, or their internal components thereof, can be administered and dosed in accordance with good medical practice, taking into account the clinical condition of the individual patient, the site and method of administration, scheduling of administration, patient age, sex, body weight and other factors known to medical practitioners.

In some embodiments, the administration is designed to supply the mammalian stressed exosomes and optionally a chemotherapeutic agent to the tumor or tumors that form the cancer that requires the effects provided by the stressed mammalian exosomes and the chemotherapeutic agent to prevent or treat the cancer and/or metastasis of a cancer.

For example, in one embodiment, a dose of the stressed mammalian exosomes may include administration of about 1×10⁵ to about 1×10¹⁹ per dose, or a daily dose, or 1×10⁷ to about 1×10¹⁷ exosomes administered per dose, or a daily dose, one or more times per day, or one or more times per week, or one or more times per month, for a period of one day, one week, two or more weeks, a 28 day regimen, or one or more months per year. In some embodiments, when the mammalian stressed exosomes and chemotherapeutic agent are dosed separately, a dosage unit may include a container for example, a vial containing 10⁷ to 10¹⁷ exosomes. In certain embodiments, a dosage unit of stressed exosomes is a vial containing 10⁷ to 10¹⁷ exosomes and at least one pharmaceutically acceptable excipient. For chemotherapeutic agent administration, the dosage of the chemotherapeutic agent may include 50 μg to about 5000 mg, or from about 75 μg to about 1000 mg, or from about 100 μg to about 500 mg, or from about 1 mg to about 300 mg administered per dose or total dose, wherein the total dose may be divided doses, the first dose administered as an initial bolus and the remainder infused over a period of time ranging from about 5 minutes to about 120 minutes. In various examples, the stressed mammalian exosomes are derived from one or more stressed cells selected from: endothelial cells, (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or cell cultured cells thereof, or their internal components thereof, or for example, any of the foregoing mammalian cell derived stressed exosomes containing miR-214, is administered prior to, concomitantly with or subsequent to the administration of the chemotherapeutic agent. In some embodiments, the mammalian stressed exosomes are dosed before the administration of the chemotherapeutic agent or concomitantly with the chemotherapeutic agent and is then administered one or more times after the administration of the chemotherapeutic agent, for example, one or more doses dosed daily, one or more times per day, one or more times per week or one or more times per month for one week to 12 months after the initial dose to treat a subject with a liver cancer or a pancreatic cancer.

In another embodiment, the stressed mammalian exosomes are derived from one or more stressed mammalian cells selected from: endothelial cells (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or cell cultured cells thereof, or their internal components thereof, or for example, any of the foregoing stressed mammalian cell derived exosomes, optionally containing miR-214 microRNA, is administered prior to, and/or concomitantly with and/or subsequent to the performance of surgical resection to remove as much cancerous tissue as reasonably feasible. In some embodiments, the stressed mammalian exosomes described herein are dosed before the performance of the surgical resection procedure. In some embodiments, the stressed mammalian exosomes described herein are dosed concomitantly with the surgical resection procedure. In some embodiments, the stressed mammalian exosomes described herein are dosed before the performance of the surgical resection procedure and the subject is then administered one or more times after the surgical resection procedure, with one or more doses of the stressed exosomes and/or the chemotherapeutic agent, for example, one or more doses dosed hourly, or one or more times per day, or one or more times per week, or one or more times per month for one week to 12 months after the initial surgical resection procedure to remove a liver cancer or a pancreatic cancer. In some related embodiments, the chemotherapeutic agent may also be dosed prior to, or concomitantly with the surgical resection procedure.

### Methods of Treatment

In one embodiment, combination therapies according to the invention comprise the administration of at least one stressed mammalian cell derived exosome containing composition or formulation of the invention and radiotherapy. In one embodiment, combination therapies according to the invention comprise the administration of at least one stressed mammalian cell derived exosome containing composition or formulation of the invention, one or more chemotherapeutic agents. In a related embodiment, the administration comprises at least one supportive care agent (e.g., at least one anti-emetic agent). In one embodiment, combination therapies according to the present invention comprise the administration of at least one stressed mammalian cell derived exosome containing composition or formulation of the invention and at least one chemotherapeutic agent. In one particular embodiment, the invention comprises the administration of at least one stressed mammalian cell derived exosome containing composition or formulation of the invention and at least one chemotherapeutic agent e.g. an anti-neoplastic agent, optionally, with one or more cancer surgical procedures, for example, a surgical resection procedure and/or one or more doses of radiation therapy. In some embodiments, the combination comprises at least one stressed exosome containing composition or formulation of the invention and at least one chemotherapeutic agent.

When a stressed mammalian cell derived exosome containing composition or formulation of the invention is used in combination with one or more chemotherapeutic agents, the dose of each active agent(s) may differ from that when the active agent is used alone. Appropriate doses will be readily appreciated by those skilled in the art. The appropriate dose of the stressed mammalian cell derived exosome containing composition or formulation(s) of the invention and the anti-neoplastic or one or more chemotherapeutic agents and the relative timings of administration will be selected in order to achieve the desired combined therapeutic effect, and are within the expertise and discretion of the attendant clinician.

Typically, any chemotherapeutic agent that has activity against a susceptible neoplasm being treated may be utilized in combination with the stressed mammalian cell derived exosome containing composition or formulation of the invention, provided that the particular agent is clinically compatible with therapy employing a stressed mammalian cell derived exosome containing composition or formulation of the invention. Typical anti-neoplastic agents useful in the present invention include, but are not limited to: alkylating agents, anti-metabolites, antitumor antibiotics, antimitotic agents, topoisomerase I and II inhibitors, hormones and hormonal analogues; retinoids, signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the compounds of the invention in the compositions and methods described above. Examples of alkylating agents include but are not limited to nitrogen mustards such as cyclophosphamides, temozolamide, melphalan, and chlorambucil; oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the compounds of the invention in the compositions and methods described above. Examples of antimetabolite anti-neoplastic agents include but are not limited to purine and pyrimidine analogues and anti-folate compounds, and more specifically, hydroxyurea, cytosine, arabinoside, ralitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the stressed exosomes of the invention in the compositions and methods described above.

Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the compounds of the invention in the compositions and methods described above. Examples of antimitotic agents include, but are not limited to, diterpenoids, vinca alkaloids, polo-like kinase (Plk) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine. Plk inhibitors are discussed further below.

Topoisomerase inhibitors include inhibitors of Topoisomerase II and inhibitors of Topoisomerase I. Topoisomerase II inhibitors, such as epipodophyllotoxins, are anti-neoplastic agents derived from the mandrake plant, e.g., Podophyllum sp., that typically affect cells in the S and G2 phases of the cell cycle by forming a ternary complex with topoisomerase II and DNA, causing DNA strand breaks. The strand breaks accumulate and cell death follows. Examples of epipodophyllotoxins include, but are not limited to, etoposide and teniposide. Camptothecins, including camptothecin and camptothecin derivatives, are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the extracellular vesicles of the invention in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the compounds of the invention in the compositions and methods described above. Examples of hormones and hormonal analogues believed to be useful in the treatment of neoplasms include, but are not limited to antiestrogens, such as tamoxifen, toremifene, raloxifene, fulvestrant, iodoxyfene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrazole, vorazole, and exemestane; progestrins such as megestrol acetate; 5α-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

Retinoid(s) are compounds that bind to and activate at least one retinoic acid receptor selected from RARα, RARβ, and RARγ and/or compounds that bind to and activate at least one of RARα, RARβ, and RARγ and also at least one retinoic X receptor (RXR), including RXRα, RXRβ, and RXRγ. Retinoids for use in the present invention typically have affinity for RAR, and particularly for RARα and/or RARβ. However, certain synthetic retinoids, such as 9-cis-retinoic acid also have affinity for both RAR and RXR. In one embodiment, the retinoid has affinity for RARα (and RARα agonist).

Signal transduction pathway inhibitors are those inhibitors which block or inhibit a chemical process which evokes an intracellular change. As used herein these changes include, but are not limited to, cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3—OH kinases, myoinositol signaling, and Ras oncogenes. Signal transduction pathway inhibitors may be employed in combination with the compounds of the invention in the compositions and methods described above.

Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1R, IR and IRR); epidermal growth factor family receptors (EGFR, ErbB2, and ErbB4); platelet derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET protooncogene.

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb™) and erlotinib (TARCEVA®). Imatinib (GLEEVEC®) is one example of a PDGFR inhibitor. Examples of VEGFR inhibitors include pazopanib, ZD6474, AZD2171, PTK787, sunitinib and sorafenib.

In one embodiment, the invention provides methods of treatment of a cancer comprising administering a therapeutically effective dose of stressed mammalian cell derived exosomes, for example, a stressed mammalian cell derived exosome containing composition or formulation in combination with an EGFR or ErbB inhibitor. In one particular embodiment, the methods of the present invention comprise administering a stressed mammalian cell derived exosome containing composition or formulation of the invention in combination with lapatinib. In one particular embodiment, the methods of the present invention comprise administering a stressed mammalian cell derived exosome containing composition or formulation of the invention in combination with trastuzumab. In one particular embodiment, the methods of the present invention comprise administering a stressed mammalian cell derived exosome containing composition or formulation of the invention in combination with erlotinib. In one particular embodiment, the methods of the present invention comprise administering a stressed mammalian cell derived exosome containing composition or formulation of the invention in combination with gefitinib.

In another embodiment, the present invention provides methods of treatment of a cancer comprising administering a stressed mammalian cell derived exosome containing composition or formulation of the invention in combination with a VEGFR inhibitor. In one particular embodiment, the methods of the present invention comprise administering a stressed exosome containing composition or formulation of the invention in combination with pazopanib.

Tyrosine kinases that are not transmembrane growth factor receptor kinases are termed non-receptor, or intracellular tyrosine kinases. Inhibitors of non-receptor tyrosine kinases are sometimes referred to as “anti-metastatic agents” and can be useful in the present invention. Targets or potential targets of anti-metastatic agents, include, but are not limited to, c-Src, Lck, Fyn, Yes, Jak, Abl kinase (c-Abl and Bcr-Abl), FAK (focal adhesion kinase) and Bruton's tyrosine kinase (BTK). Non-receptor kinases and agents, which inhibit non-receptor tyrosine kinase function, are described in Sinha, S, and Corey, S. J., (1999) J. Hematother. Stem Cell Res. 8:465-80; and Bolen, J. B. and Brugge, J. S., (1997) Annu. Rev. of Immunol. 15:371-404.

SH2/SH3 domain blockers are agents that disrupt SH2 or SH3 domain binding in a variety of enzymes or adaptor proteins including, but not limited to, PI3-K p85 subunit, Src family kinases, adaptor molecules (Shc, Crk, Nck, Grb2) and Ras-GAP. Examples of Src inhibitors include, but are not limited to, dasatinib and BMS-354825 (J. Med. Chem. (2004) 47:6658-6661).

Inhibitors of serine/threonine kinases may also be used in combination with stressed mammalian cell derived exosome containing composition or formulation of the invention in any of the compositions and methods described above. Examples of serine/threonine kinase inhibitors that may also be used in combination with a mammalian cell derived exosome containing composition or formulation of the invention include, but are not limited to, polo-like kinase inhibitors (Plk family e.g., Plk1, Plk2, and Plk3), which play critical roles in regulating processes in the cell cycle including the entry into and the exit from mitosis; MAP kinase cascade blockers, which include other Ras/Raf kinase inhibitors, mitogen or extracellular regulated kinases (MEKs), and extracellular regulated kinases (ERKs); Aurora kinase inhibitors (including inhibitors of Aurora A and Aurora B); protein kinase C (PKC) family member blockers, including inhibitors of PKC subtypes (alpha, beta, gamma, epsilon, mu, lambda, iota, zeta); inhibitors of kappa-B (IkB) kinase family (IKK-alpha, IKK-beta); PKB/Akt kinase family inhibitors; and inhibitors of TGF-beta receptor kinases. Other examples of serine/threonine kinase inhibitors are known in the art. In another embodiment, the present invention provides methods of treatment of a cancer comprising administering a stressed mammalian cell derived exosome containing composition or formulation of the invention in combination with a Plk inhibitor.

Urokinase, also referred to as urokinase-type Plasminogen Activator (uPA), is a serine protease. Activation of the serine protease plasmin triggers a proteolysis cascade which is involved in thrombolysis or extracellular matrix degradation. Elevated expression of urokinase and several other components of the plasminogen activation system have been correlated with tumor malignancy including several aspects of cancer biology such as cell adhesion, migration and cellular mitotic pathways as well. Inhibitors of urokinase expression may be used in combination with the mammalian cell derived exosome containing composition or formulation of the invention in the compositions and methods described above.

Inhibitors of Ras oncogene may also be useful in combination with the mammalian cell derived exosome containing composition or formulation of the invention. Such inhibitors include, but are not limited to, inhibitors of farnesyltransferase, geranyl-geranyl transferase, and CAAX proteases as well as anti-sense oligonucleotides, ribozymes and immunotherapy. Such inhibitors have been shown to block Ras activation in cells containing mutant Ras, thereby acting as antiproliferative agents.

Inhibitors of kinases involved in the IGF-1R signaling axis may also be useful in combination with the stressed CEC derived exosome containing composition or formulations of the present invention. Such inhibitors include but are not limited to inhibitors of JNK1/2/3, PI3K, AKT and MEK, and 14.3.3 signaling inhibitors.

Cell cycle signaling inhibitors, including inhibitors of cyclin dependent kinases (CDKs) are also useful in combination with the stressed mammalian cell derived exosome containing composition or formulation of the invention in the compositions and methods described above. Examples of cyclin dependent kinases, including CDK2, CDK4, and CDK6 and inhibitors for the same are described in, for instance, Rosania G. R., et al., Exp. Opin. Ther. Patents (2000) 10:215-230.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the stressed CEC derived exosome containing composition or formulations of the invention. For example, anti-VEGF antibodies, which do not recognize VEGFR (the receptor tyrosine kinase), but bind to the ligand; small molecule inhibitors of integrin (alphav beta3) that inhibit angiogenesis; endostatin and angiostatin (non-RTK) may also prove useful in combination with the stressed CEC derived exosome containing composition or formulations of the invention. One example of a VEGFR antibody is bevacizumab (AVASTIN®). Inhibitors of phosphatidyl inositol-3—OH kinase family members including blockers of PI3-kinase, ATM, DNA-PK, and Ku may also be useful in combination with the present invention.

In some embodiments, stressed mammalian cell derived exosome containing composition or formulation of the invention may be administered before, concomitantly or after the administration of the following exemplary chemotherapeutics: inhibitors of topoisomerase I and II activity, such as camptothecin, drugs such as irinotecan, topotecan and rubitecan, alkylating agents such as temozolomide and DTIC (dacarbazine), and platinum agents like oxaliplatin, cisplatin, cisplatin-doxorubicin-cyclophosphamide, carboplatin, and carboplatin-paclitaxel, doxorubicin, doxorubicin-cyclophosphamide, capecitabine, cyclophosphamide-methotrexate-5-fluorouracil, docetaxel, paclitaxel, 5-fluoracil-epirubicin-cyclophosphamide, paclitaxel, vinorelbine, etoposide, pegylated liposomal doxorubicin and topotecan. In some embodiments, the chemotherapeutic useful to be used in combination with the exosome compositions of the present invention, may include: Platinum agents: cisplatin, carboplatin, oxaliplatin, Vinca alkaloids: vincristine, vinblastine, Taxanes: paclitaxel, docetaxel, Epothilones: ixabepalone, and bortezomib, thalidomide, and lenolidamide.

In various embodiments, chemotherapeutic agents in the form of inhibitors of immune checkpoint PD-1, CTLA-4, PD-L1 and PD-L21 pathways may also be useful in combination with the stressed mammalian cell derived exosome containing composition or formulation of the invention. Such inhibitors include, but are not limited to antibodies, such as Nivolumab (also known as ONO-4538, BMS-936558 or MDX-1106) is a genetically engineered, fully human immunoglobulin (Ig) G4 immune checkpoint inhibitor specifically targeting for human PD-1. The antibody binds to PD-1 with high affinity, thereby attenuating inhibitory signals and enhancing the host antitumor immune responses. Also exemplified is Pembrolizumab (MK-3475), a humanized IgG4 monoclonal antibody blocking the interaction of PD-1 on T cells with its ligands, is believed to reactivate antitumor immunity. Also exemplified is Pidilizumab (CT-011) is a humanized IgG1 kappa recombinant monoclonal antibody against PD-1. In preclinical studies, pidilizumab was demonstrated to inhibit cancer cells survival. Also exemplified are the anti-PD ligand 1 (PD-L1) mAbs Atezolizumab and Durvalumab which showed preliminary antitumor activity in multiple solid cancers before their recent FDA approval for multiple cancers.

The PD-1/PD-L1 pathway has a crucial role in regulating immunosurveillance for tumors. PD-1 can interfere with TCR/CD28 signals to suppress the immune responses of T-cell help (Tc1/Th1 skewing) in the tumor microenvironment through the PD-1/SHP-2/p-STAT1/T-bet axis. Tumor cells expressing PD-1 can limit the activity of tumor antigens (TA)-specific CD8+ T cells, which reinforces their growth and invasiveness. PD-1 is upregulated by dysfunctional TA-specific CD8+ T cells both in vitro and in vivo, and PD-1 blockade enhances TA-specific T cell responses and inhibits tumor growth or partial tumor regression. PD-1 blockade also increases T-cell migration to tumors by elevating IFN-γ inducible chemokines, which augments T-cell-mediated antitumor responses. In addition, the majority of TILs predominantly express high levels of PD-1 and are thought to be correlated with an “exhausted” phenotype and impaired antitumor immune responses. This “exhausted” phenotype is marked by decreased T cell proliferation, poor cytolytic activity, and low production of type I cytokines.

PD-L1 and PD-L2 expression are up-regulated in a variety of human cancer types. PD-L1 is frequently expressed in several types of solid tumor cells, whereas PD-L2 is highly expressed in certain subsets of B cell lymphomas. Expression of PD-L1 protein significantly correlates with the levels of elevated TILs, which is associated with cancer metastasis. Transgenic expression of PD-L1 in immunogenic tumor cells confers them a potent escaping from host T cell immunity and markedly enhances their invasiveness in vivo. PD-L1 is also upregulated in tumors by activation of key signaling pathways including PI3K, STAT3, IFN-γ and so on. Latent membrane protein 1 (LMP1) and IFN-γ upregulate PD-L1 through STAT3, AP-1, and NF-κB pathways, which promotes progression of nasopharyngeal carcinoma (NPC) and pancreatic cancer. The activation of MAPK promotes PD-L1 expression that is transcriptionally modulated by c-Jun and augmented by STAT3. Similarly, PD-L2 expression is observed in a subset of tumor types but its role in cancer is far less prevalent than PD-L1. PD-L2 expression in pulmonary squamous cell carcinoma is associated with an increased number of CD8+TILs and proto-oncogene MET protein overexpression.

In each of the aforementioned methods of treatment using the illustrated chemotherapeutic agents described above, the stressed mammalian cell derived exosomes are stressed CEC derived exosomes, and/or stressed endothelial cell progenitor cell derived exosomes, and wherein the stressed CEC derived exosomes, and/or stressed endothelial cell progenitor cell derived exosomes are derived from human CECs and human endothelial cell progenitors, obtained from a human sample, or cultured under in-vitro conditions known to those skilled in the art, wherein the stressed mammalian cells used, whether CECs or endothelial cell progenitor cells are stressed by hypoxia and/or depriving said mammalian cells of oxygen and glucose, for at least one hour during culturing these cells to produce stressed CEC derived exosomes, and/or stressed endothelial cell progenitor cell derived exosomes.

The present invention has identified several unexpected findings as illustrated in the figures and detailed description. One such unexpected finding includes the discovery that when cancer cells are contacted with stressed mammalian cell derived exosomes, for example, stressed CEC derived exosomes, and a chemotherapeutic agent, the cancer cells become sensitized to the effects of the administered chemotherapeutic agent. The present invention therefor enables the chemotherapeutic agent to be administered or used in a dosage regimen at a concentration that is lower than the standard therapeutic dosage for administering the chemotherapeutic agent to a similar subject having the same or similar cancer without administration of the stressed exosomes. Alternatively, the present invention therefor enables the chemotherapeutic agent to be administered or used in a dosage regimen at a concentration that is higher than the standard therapeutic dosage for administering the chemotherapeutic agent to a similar subject having the same or similar cancer with administration of the stressed exosomes.

Those of ordinary skill in the art understands and administers chemotherapeutic agents based on the directions of a regulatory agency and the directions provided on the label associated with each approved chemotherapeutic agent. Accordingly, the combination comprising a stressed mammalian cell derived exosomes containing composition (for example, stressed endothelial progenitor cell exosomes or stressed CEC-derived exosomes), and one or more chemotherapeutic agents permits dosing the chemotherapeutic agent(s) of the combination at sub-therapeutic doses or at higher doses than the doses which are approved for use in a specific cancer patient population. Since the dose of the chemotherapeutic agent in the combination is administered at a lower or higher concentration or amount than the dose approved for use in a specific cancer population based on a similar weight, age, type and stage of cancer, (i.e. a maximally tolerated dose, optionally titrated to avoid unwanted side effects individually catered for the specific patient) then the subject is expected to tolerate the chemotherapeutic agent used in the combination for a longer period compared to the dose of the chemotherapeutic agent when dosed alone. As used herein, approved doses of chemotherapeutic doses are doses that are approved by a regulatory agency for use by a specific patient population for a cancer treatment, which is typically recited in the label accompanying each approved chemotherapeutic agent for use in their respective cancer treatment.

Another unexpected finding is that since the combination comprising a stressed mammalian cell derived exosomes containing composition (for example, stressed endothelial cell progenitor derived exosomes or stressed CEC-derived exosomes) sensitizes the tumor or cancer cells to the effects of the chemotherapeutic agent, then the administration of the combination permits a lower dosage of the chemotherapeutic agent which may then reduce, ameliorate or eliminate one or more side effects (for example, neurotoxicity) associated with multiple administrations of the chemotherapeutic agent when used alone in a population of similar subjects treated for the same or similar cancer.

Another unexpected finding is that since the combination comprising a stressed mammalian cell derived exosomes containing composition (for example, stressed endothelial cell progenitor derived exosomes or stressed CEC-derived exosomes) sensitizes the tumor or cancer cells to the effects of the chemotherapeutic agent, then the administration of the combination permits a higher dosage of the chemotherapeutic agent which may then reduce, ameliorate or eliminate one or more side effects (for example, neurotoxicity) associated with multiple administrations of the chemotherapeutic agent when used alone in a population of similar subjects treated for the same or similar cancer.

Methods for determining the presence or absence of a cancer, of the efficacy of any of the treatments described herein are well known to oncology professionals in the art. For example, the same method of assessment and the same technique should be used to characterize each identified and reported cancer lesion or population of circulating cancer cells at baseline and during follow-up. Imaging-based evaluation is preferred to evaluation by clinical examination when both methods have been used to assess the antitumor effect of a treatment.

Clinical examination. Clinically detected lesions will only be considered measurable when they are superficial (e.g., skin nodules and palpable lymph nodes). For the case of skin lesions, documentation by color photography—including a ruler to estimate the size of the lesion—is recommended.

X-ray. Lesions on x-ray screening are acceptable as measurable lesions when they are clearly defined and surrounded by aerated lung or tissue. However, CT is preferable.

CT and MRI. CT and MRI are the best currently available and most reproducible methods for measuring target lesions selected for response assessment. Conventional CT and MRI should be performed with contiguous cuts of 10 mm or less in slice thickness. Spiral CT should be performed by use of a 5-mm contiguous reconstruction algorithm; this specification applies to the tumors of the chest, abdomen, and pelvis, while head and neck tumors and those of the extremities usually require specific protocols.

Ultrasound. When the primary end point of the study is objective response evaluation, ultrasound should not be used to measure tumor lesions that are clinically not easily accessible. It may be used as a possible alternative to clinical measurements for superficial palpable lymph nodes, subcutaneous lesions, and thyroid nodules. Ultrasound might also be useful to confirm the complete disappearance of superficial lesions usually assessed by clinical examination.

Endoscopy and laparoscopy. The utilization of these techniques for objective tumor evaluation has not yet been fully or widely validated. Their uses in this specific context require sophisticated equipment and a high level of expertise that may be available only in some centers. Therefore, utilization of such techniques for objective tumor response should be restricted to validation purposes in specialized centers. However, such techniques can be useful in confirming complete histopathologic response when biopsy specimens are obtained.

Tumor markers. Tumor markers alone cannot be used to assess response. However, if markers are initially above the upper normal limit, they must return to normal levels for a patient to be considered in complete clinical response when all tumor lesions have disappeared. Specific additional criteria for standardized usage of prostate-specific antigen and CA (cancer antigen) 125 response in support of clinical trials are being validated.

Cytology and histology. Cytologic and histologic techniques can be used to differentiate between partial response and complete response in rare cases (e.g., after treatment to differentiate between residual benign lesions and residual malignant lesions in tumor types such as germ cell tumors). Cytologic confirmation of the neoplastic nature of any effusion that appears or worsens during treatment is required when the measurable tumor has met criteria for response or stable disease. Under such circumstances, the cytologic examination of the fluid collected will permit differentiation between response or stable disease (an effusion may be a side effect of the treatment) and progressive disease (if the neoplastic origin of the fluid is confirmed). New techniques to better establish objective tumor response will be integrated into these criteria when they are fully validated to be used in the context of tumor response evaluation.

As used herein, the forms of cancer treatable using the stressed exosomes, for example, stressed exosomes derived from CECs which were subjected to hypoxia and/or OGD include: breast cancer (e.g. ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), thyroid cancer, endocrine system cancer, brain cancer, cervical cancer, colon cancer, head & neck cancer, liver cancer, kidney cancer, non-small cell lung cancer, mesothelioma, stomach cancer, uterine cancer, Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, prostate cancer, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, undifferentiated cell leukemia, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum, hepatocellular carcinoma (HCC), intrahepatic cholangiocarcinoma, angiosarcoma and hemangiosarcoma, hepatoblastoma, hemangiomas, hepatic adenomas, focal nodular hyperplasia, pancreatic adenocarcinoma, adenosquamous carcinomas, squamous cell carcinomas, signet ring cell carcinomas, undifferentiated carcinomas, carcinoma of the ampulla of Vater, undifferentiated carcinomas with giant cells, serous cystic neoplasms, mucinous cystic neoplasms, intraductal papillary mucinous neoplasms, and solid pseudopapillary neoplasms and secondary cancers such as metastatic cancer or refractory or advanced cancers.

### Kits

In some embodiments, the present disclosure provides kits for the treatment and prevention of a cancer. In some embodiments, the kit of the present disclosure comprises stressed mammalian exosomes. In some embodiments, the stressed mammalian exosomes are derived from for example, endothelial cells (for example, cerebral endothelial cells (CEC), brain microvascular endothelial cells (BMVEC), Primary Human Brain Microvascular Endothelial Cells (PHBMVEC) (for example, ACBRI 376), endothelial progenitor cells, AG-133/CD-133+ cells and the like), glial cells, astrocytes, neurons, oligodendrocytes, spindle neurons, microglia, or mastocytes, or cell cultured cells thereof, or their internal components thereof, or for example, any of the foregoing stressed mammalian cell derived exosomes. In some embodiments, the stressed mammalian cell derived exosomes may contain one or more of: miR-19a, miR-21, and miR-146a microRNA. In various embodiments, the kits of the present disclosure contain stressed mammalian exosomes derived from the above referenced mammalian cells, and wherein the exosome composition contains one or more of miR-19a, miR-21, and miR-146a microRNA. The kit of the present disclosure can include one or more doses of stressed mammalian exosomes in combination with a therapeutic dose of one or more chemotherapeutic agents, in the same or separate compositions. In some embodiments, the kit of the present disclosure. The kit of the present disclosure can include one or more doses of stressed mammalian exosomes in combination with a therapeutic dose of a chemotherapeutic agent, in the same or separate compositions. In some embodiments, the kit of the present disclosure can include one or more doses of stressed mammalian exosomes in combination with a surgical device useful in the performance of a cancer tissue resection surgery procedure. In various embodiments, the kit of the present disclosure also includes a package insert comprising instructions for using the stressed mammalian exosomes and a chemotherapeutic agent.

While some embodiments have been particularly shown and described with reference to the foregoing preferred and alternative embodiments, it should be understood by those skilled in the art that various alternatives to the embodiments described herein may be employed in practicing the invention without departing from the spirit and scope of the invention as defined in the following claims. It is intended that the following claims define the scope of the invention and that the methods, systems, and compositions within the scope of these claims and their equivalents be covered thereby. This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### Examples

### Example 1. Hypoxia CEC Exosomes Collection

Grow human primary CEC cells (Cell Systems: ACBRI 376) in 75 cm² flasks in complete growth medium (Cell Systems: 4Z0-500) until the cells reach 80-90% confluency. Pre-warm PBS at 37° C. Wash cells with PBS. Add 10 ml pre-warmed serum-free medium (Cell Systems: SF-4Z0-500) to each flask. Incubate half of the flasks in hypoxia chamber with 0 oxygen for 24 hours. Then transfer the flasks into regular incubator for another 24 hours before collecting the medium. Add 10 ml of serum-free medium, incubate another 48 hours and collect the conditioned medium. Incubate the other half of the flasks in regular CO2 incubator and collect conditioned medium every 48 hours. When colleting the conditioned growth medium, spin down at 4,000 rpm for 15 min at 4° C. Label either hypoxia exosome medium or naïve exosome medium correctly, and freeze the medium at about −20° C. until ready to isolate exosomes.

### Example 1.1—Isolation and Use of Exosomes Isolated from Oxygen and/or Glucose Deprived Cerebral Endothelial Cells (CECs)

Extracellular vesicles (EV) are lipid bound vesicles that are excreted by almost all cells into the extracellular space. EVs vary in size and exosomes are a type of EV enclosed in a single outer membrane that range, in size, between 30 to 150 nm in diameter (Doyle 2019 Cell). Exosomes are found in all body fluids including blood, cerebrospinal fluid, urine, lymph, bile and breast milk. EVs, in general, and exosomes, in particular, contain a cargo of nucleic acids, proteins and lipids that are derived from the parent cell. EVs have been shown to transfer their cargo from one cell to another, mediate cell to cell communication and affect the biology of recipient cells in a wide variety of settings. In tissue culture systems, EVs can play a role in antigen-presentation and immune and inflammation regulation. EVs are postulated to play protean roles in cancer biology. In both in vitro and in vivo models, they play a role in cell cycle and proliferation, angiogenesis, epithelial-mesenchymal transition (EMT) and metastatic niche promotion (Kalluri Science 2020). In the present disclosure, studies undertaken demonstrate that exosomes derived from human cerebral endothelial cells exposed to stress, for example, oxygen and/or glucose deprivation, produce exosomes that when administered to a subject with cancer, demonstrate potent anticancer activity across liver cancers and pancreatic cancers, when administered alone, or when transfected to produce increased miR-214.

A. Exosomes Derived from Oxygen Glucose Deprivation (OGD) Challenged Human Cerebral Endothelial Cells (OGD-Exos) Decrease the Viability of Liver Cancer Cells

Based on preliminary research, we hypothesized that under duress, cells and cell lines might generate exosomes that are deleterious specifically to malignant cells and tissue. We also speculated that exosomes could be loaded with specific microRNA (mi-RNA) that might further enhance anti-cancer activity and specificity. We initiated studies with human cerebral endothelial cells (HCEC) because in vitro, endothelial cells would be able to release exosomes directly into the systemic circulation where they could travel to cancer sites. The initial mechanism of cellular stress we chose for the HCEC cells was oxygen-glucose deprivation (OGD). HCEC cells were exposed to OGD deprivation for a period of four hours and then exosomes were harvested and purified (See methods). HCEC cells exposed to OGD deprivation for longer than four hours started to lose viability. The “4 h-OGD-exosomes” were then tested for their effect on the viability of hepatocellular cancer cell lines, HepG2 and Hep3B, and on the benign liver cell line, THLE-2. To assay the cell viability of target cells, we used the MTT (3-(4,5-Dimethylthiazol-2-yl)-2,5-diphenyltetrazolium bromide) assay (See Methods). All cancer lines used in all of the studies are outlined in Table 1.

TABLE 1
Cancer and benign cell lines used as targets for OGD-Exo and
Hypo-Exo

| | | Source | |
|---|---|---|---|
| Description | Abbreviation | (Company) | Catalog Number |
| Hepatocellular Carcinoma | HepG2 | American Type | HB-8065 |
| HepG2 | | Culture Collection | |
| | | (ATCC) | |
| Hepatocellular Carcinoma | Hep3B | American Type | HB-8064 |
| Hep3B | | Culture Collection | |
| | | (ATCC) | |
| Liver Epithelial Cells; | THLE-02 | American Type | CRL-2706 |
| Normal | | Culture Collection | |
| THLE-02 | | (ATCC) | |
| Pancreatic Carcinoma | PANC-1 | American Type | CRL-1469 |
| PANC-1 | | Culture Collection | |
| | | (ATCC) | |
| Lung Carcinoma | A549 | American Type | CCL-185 |
| A549 | | Culture Collection | |
| | | (ATCC) | |
| Prostate Carcinoma | PC3 | American Type | CRL-1435 |
| PC3 | | Culture Collection | |
| | | (ATCC) | |
| Breast Adenocarcinoma | MDA-MB-468 | American Type | HTB-132 |
| MDA-MB-468 | | Culture Collection | |
| | | (ATCC) | |
| Breast Adenocarcinoma | MDA-MB-231 | American Type | HTB-26 |
| MDA-MB-231 | | Culture Collection | |
| | | (ATCC) | |
| Colorectal Carcinoma | HCT-116 | American Type | CCL-247 |
| HCT-116 | | Culture Collection | |
| | | (ATCC) | |
| Ovarian Carcinoma | OVCAR-3 | American Type | HTB-161 |
| OVCAR-3 | | Culture Collection | |
| | | (ATCC) | |
| Ovarian Carcinoma | A2780 Parental | SigmaAldrich- | 93112519 |
| A2780 Parental | | ECACC | |
| Ovarian Carcinoma | A2780 Cisplatin | SigmaAldrich- | 93112517 |
| A2780 Cisplatin | | ECACC | |
| Ovarian Carcinoma | mCherry- | Wayne State | Established from |
| F2 | OCSC1-F2 | University- | patient with serous |
| | | Dr. Mor Lab | epithelial ovarian |
| | | | cancer |
| Fibroblasts, Mouse | NIH-3T3 | American Type | CRL-1658 |
| NIH-3T3 | | Culture Collection | |
| | | (ATCC) | |

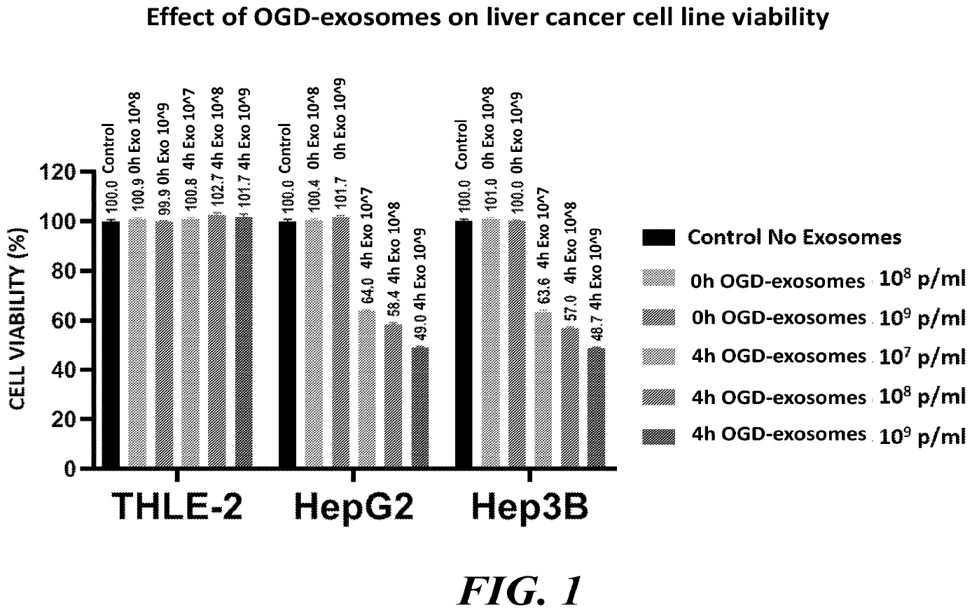

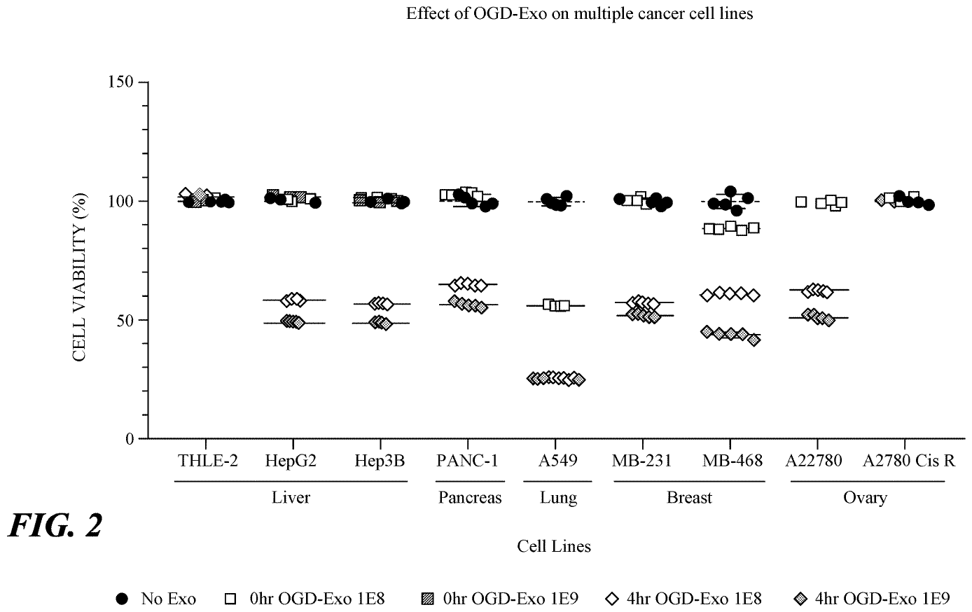

4 h-OGD-Exo were used at concentrations of 10⁷ particles/ml (p/ml), 10⁸ p/ml and 10⁹ p/ml. Treatment of the Hep3B and HepG2 HCC cancer cell lines, described above, with OGD-exosomes from HCEC cells exposed to oxygen-glucose deprivation for four hours significantly reduced cancer cell viability in a dose dependent manner, but did not significantly affect non-cancer viability (FIG. 1). At the highest concentration of 10⁹ p/ml, 4 h-OGD-Exo significantly reduced HepG2 and Hep3B cancer cell viability by 51% compared to HCC cells without exosome treatment. Exosomes from HCEC cells with no exposure to OGD (0h-OGD-Exo) have no effect on HCC cell viability. Critically, the 4 h-OGD-Exo have no impact on the viability of the non-cancerous THLE-2 liver cell line.

We then tested OGD-Exo against non-hepatic cancer lines (Table 1) and again found 4 h-OGD-Exos significantly reduced cell viability in a dose-dependent manner. At the highest dose of 10⁹ p/ml, 4 h-OGD-Exo reduced the viability of human pancreas (PANC-1), lung (A549) and breast cancer (MDA-MB-468: MDA-MB-231) cell lines by 44%, 75% and 48-56% respectively (FIG. 2). The 4 h-OGD-Exo data is shown for 10⁸ p/ml and 10⁹ p/ml. Individual dots represent individual wells. No exosomes were effective against benign THLE-2 liver cells and the multi-resistant ovarian line A2780cis. Again exosomes from HCEC cells without OGD exposure (0h-OGD-Exo) had no effect on cancer cells. These data indicate that OGD-Exos demonstrate a significant anti-cancer effect against an array of cancer types that allows for large scale experimental and commercial production.

The effect of OGD-Exo derived from human cerebral endothelial cells on cancer cells can be augmented with HCEC exosomes enriched with miR-214.

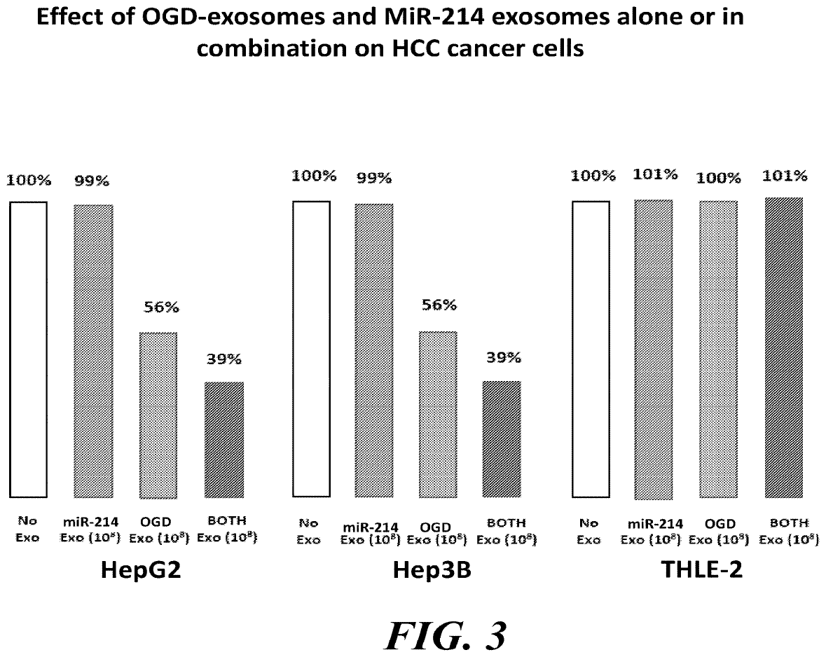

MicroRNAs are small, non-coding RNAs that negatively regulate gene expression at the post-transcriptional level. MiR-214 is reported to function as a tumor suppressor in HCC and is under expressed in HepG2 and Hep3B cells compared to THLE-2 cells (Semaan Oncotarget 2021). We hypothesized that OGD-Exo from HCEC cells might work synergistically or additively with miR-214 enriched exosomes. MiR-214 enriched exosomes were generated by transfecting miR-214 encoding pre-RNA into parental HCEC cells and enrichment of miR-214 in the resulting exosomes was confirmed with RT-PCR (See Methods). The combination of OGD-Exo and HCEC-m214-Exo, both at a concentration of 10⁸ p/ml, was significantly better against HepG2 and Hep3B cells than either type of exosome alone (FIG. 3). HCEC-m214-Exo alone had no effect against any cell line. However, the combination of OGD-Exo and HCEC-m214-Exo decreased cell viability of both HepG2 and Hep3B cells by 61% compared to 44% with 4 h-OGD-Exo alone. No exosomes alone or in combination had any efficacy against THLE-2 cells.

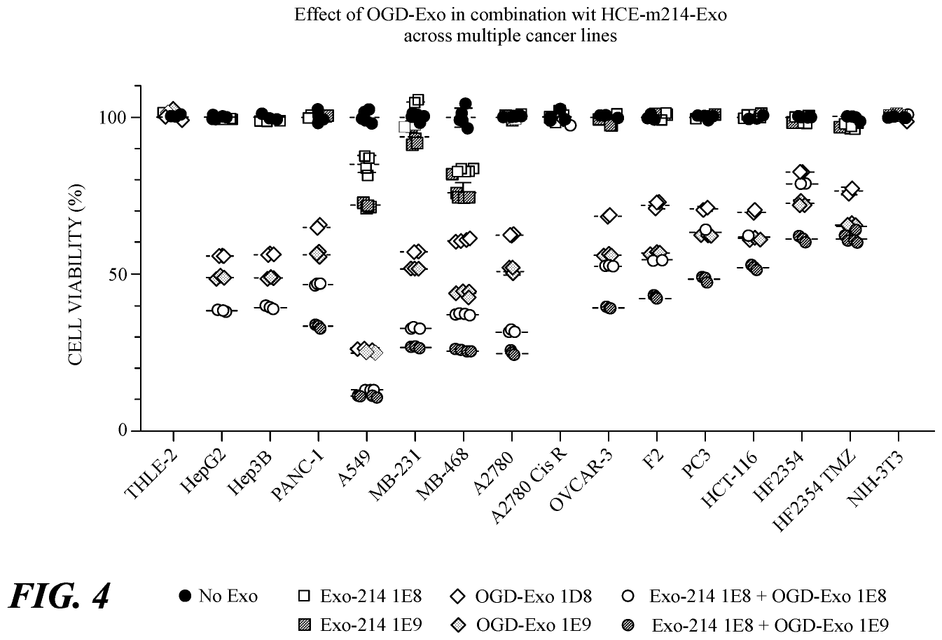

Data in FIG. 4 demonstrate the efficacy of OGD-Exo and the combination of OGD-Exo and HCEC-m214-Exo across a broad array of cancer lines. With the exception of the HCC groups, exosomes were tested at concentrations of 10⁸ exosomes or particles (p)/mL (p/ml) and 10⁹ p/ml. Cancer lines studied include (Table 1) pancreas (PANC-1), lung (A549), breast (MB-231, MB-468), ovarian (A2780, A2780cis, OVCAR, F2), prostate (PC3), colorectal (HCT-116) and glioblastoma multiforme (HF2354, HF2354 TMZ). Again, as noted previously, OGD-Exo are effective against across all cancer types selected. OGD-Exo are not effective against non-cancerous THLE-2 cells and NIH-3T3 fibroblasts. The combination of OGD-Exo and HCEC-m214-Exo is typically more effective than OGD-Exo alone.

OGD-Exo have anti-cancer activity against primary liver cancer cells from human patients and this activity is enhanced by chemotherapeutic agents.

We then examined whether OGD-Exo demonstrate anti-cancer activity against primary liver cancer cells taken from patients who underwent liver transplant with the diagnosis of hepatocellular carcinoma. Exosomes were tested against primary liver cancer cells taken from the patient's tumor as well as non-malignant, normal liver cells taken from a distant site in the explant liver away from the tumor. OGD-Exo were tested along with HCEC-m214-Exo and also with the chemotherapeutic agents, oxaliplatin and Sorafenib. Sorafenib is a small molecule multi-kinase inhibitor approved for therapy of HCC in humans. OGD-Exo were used at a dose of 10⁸ p/ml to avoid excessive killing with OGD-Exo alone. Exosomes were tested against patient 11 (Pt.011) and patient 12 (Pt.012). Doses of oxaliplatin and Sorafenib were chosen that did not produce maximal killing when used alone and were 0.06125 UM for the former and 1.20 μM for the latter.

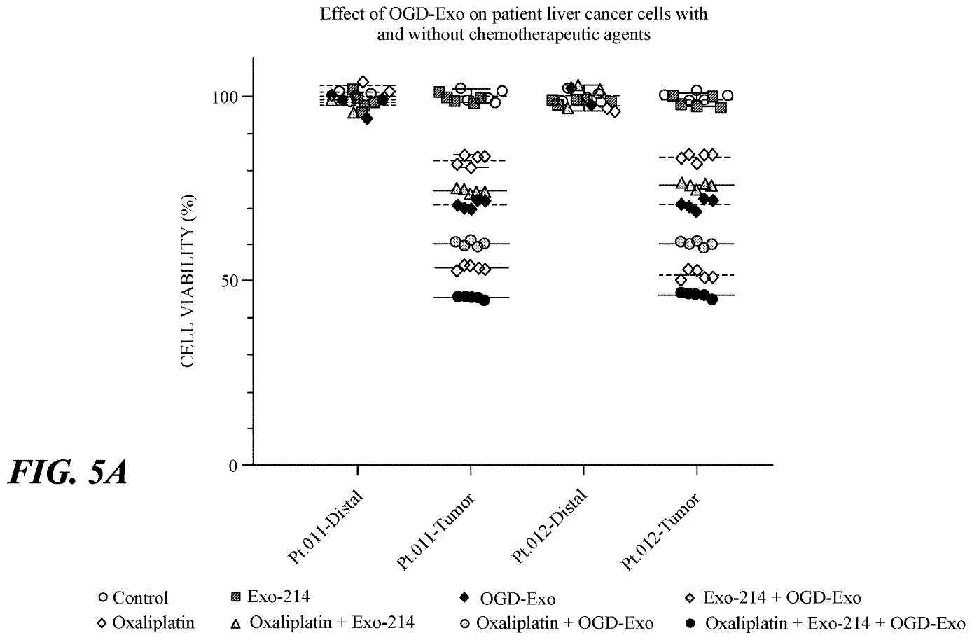

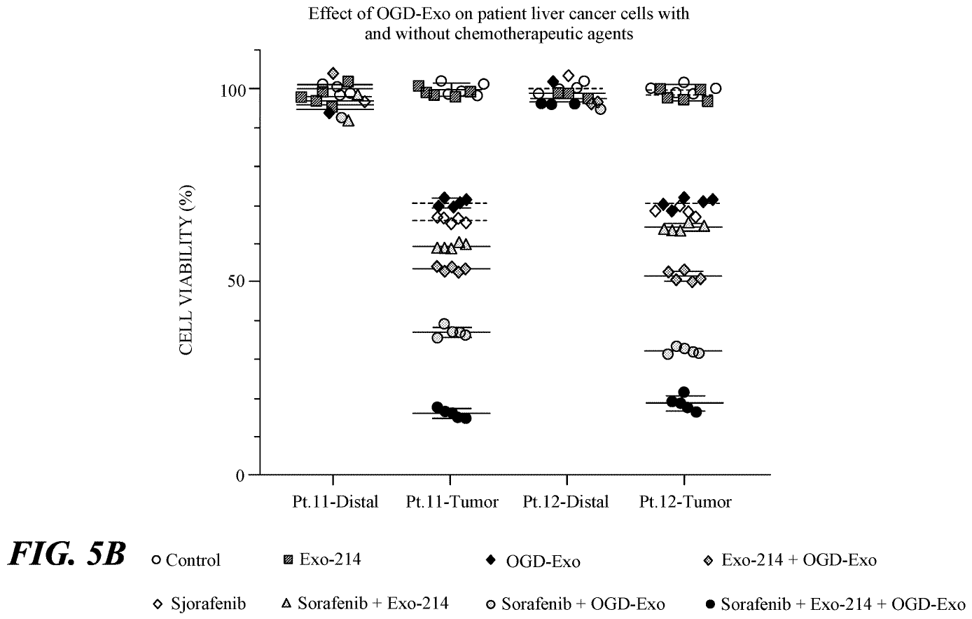

OGD-Exo at a dose of 10⁸ p/ml led to 30-35% decrease in viability of primary liver cancer cells with no effect non-cancerous liver cells (FIG. 5). The combination of OGD-Exo and HCEC-m214-Exo decreased cancer cell viability by 46-48%. The combination of OGD-Exo and oxaliplatin decreased HCC viability by 40% again compared to 30-35% for OGD-Exo alone and compared to approximately 20% for oxaliplatin alone (FIG. 5A). The combination of OGD-Exo and Sorafenib decreased viability by 63-67% again compared to 30-35% for OGD-Exo alone and compared to 42-44% for Sorafenib alone (FIG. 5B). The most potent combinations were OGD-Exo with HCEC-m214-Exo and the chemotherapeutic agent all together. With oxaliplatin this led to a decrease in viability of 54-55% (FIG. 5A) and with Sorafenib (FIG. 5B), the decrease in HCC viability was 80%. The enhanced killing effect of OGD-Exo and HCEC-m214-Exo with chemotherapeutic agents is specific to primary tumor HCC cells, because the treatment had no effect on the viability of non-neoplastic liver cells derived from liver tissue distal to the cancer of HCC patients. These in vitro data suggest that the combination of OGD-Exo and HCEC-m214-Exo can robustly sensitize the anti-cancer effect of oxaliplatin and Sorafenib on HCC cells.

B. Exosomes Derived from Human Cerebral Endothelial Cells (HCEC) Challenged with Hypoxia Reduce the Viability of Cancer Cells

We determined that HCEC cells exposed to hypoxia alone were viable for a longer period of time than HCEC cells exposed to oxygen and glucose deprivation. We found HCEC cells were viable after 24 hours of hypoxia exposure whereas cells had difficult tolerating OGD for more than four hours. We hypothesized that exosomes form HCEC cells exposed to hypoxia alone (Hypo-Exo) for more prolonged periods of time might exhibit more potent and selective anti-cancer activity.

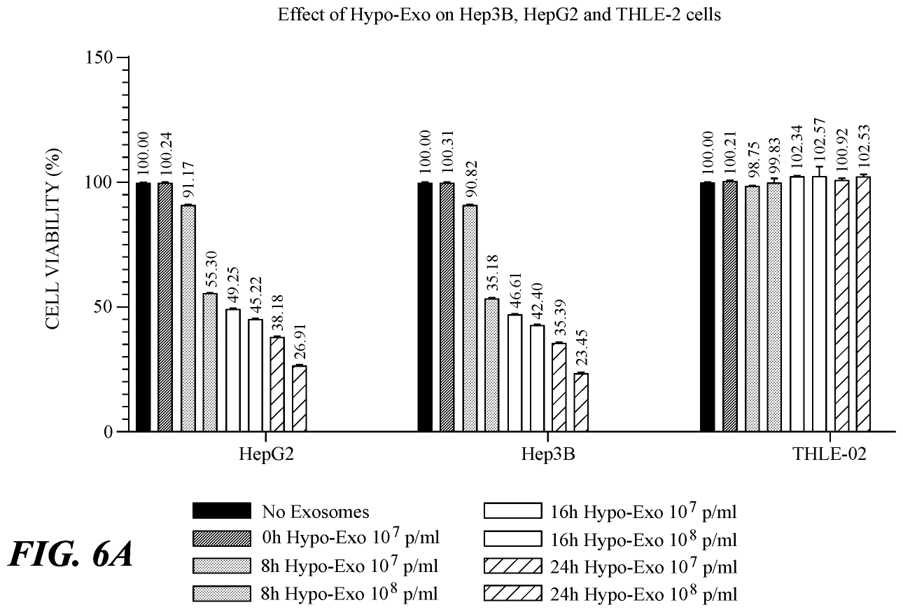

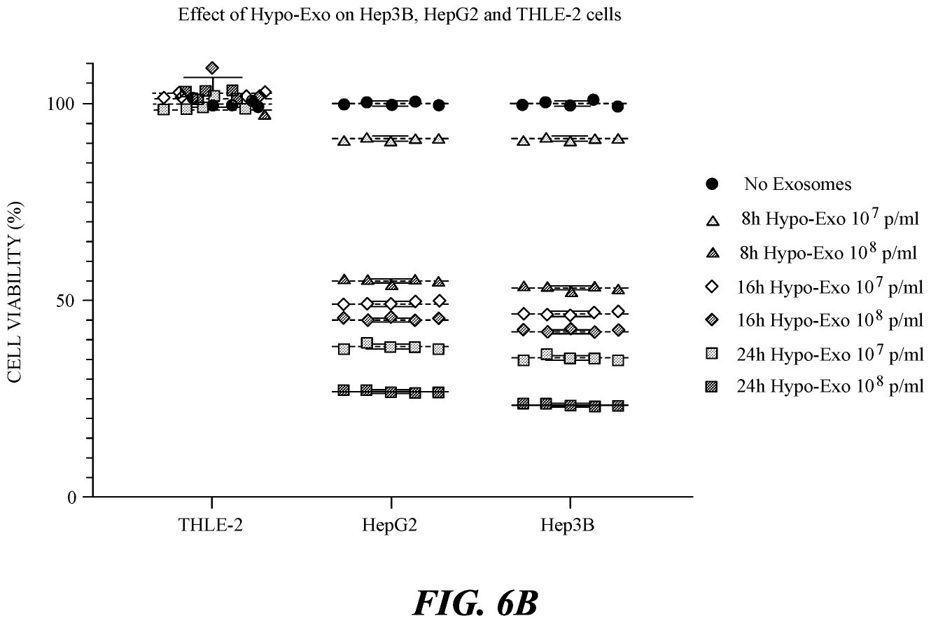

We isolated exosomes from HCEC cells exposed to varying lengths of hypoxia, from eight to 24 hours, and tested these Hypo-Exo against HCC cells lines for five days (See methods). As shown in FIG. 6, the longer the HCEC cells are exposed to hypoxia, the more potent the resulting Hypo-Exo are in reducing the viability of HCC cancer cells. The same data is shown in two formats (FIG. 6A and FIG. 6B). After 24 hours of hypoxia, the HCEC cells generated 24 h-Hypo-Exo that, at 10⁸ p/ml, resulted in decreased viability of HepG2 cells by 73% and Hep3B cells by 76%. Exosomes from HCEC cells not exposed to hypoxia (0h-Hypo-Exo) had no effect on HCC cell lines and no exosomes affected the benign THLE-2 liver cells.

### C. Hypo-Exo Demonstrate Anti-Cancer Activity Against an Array of Non-Hepatic Cancer Cells

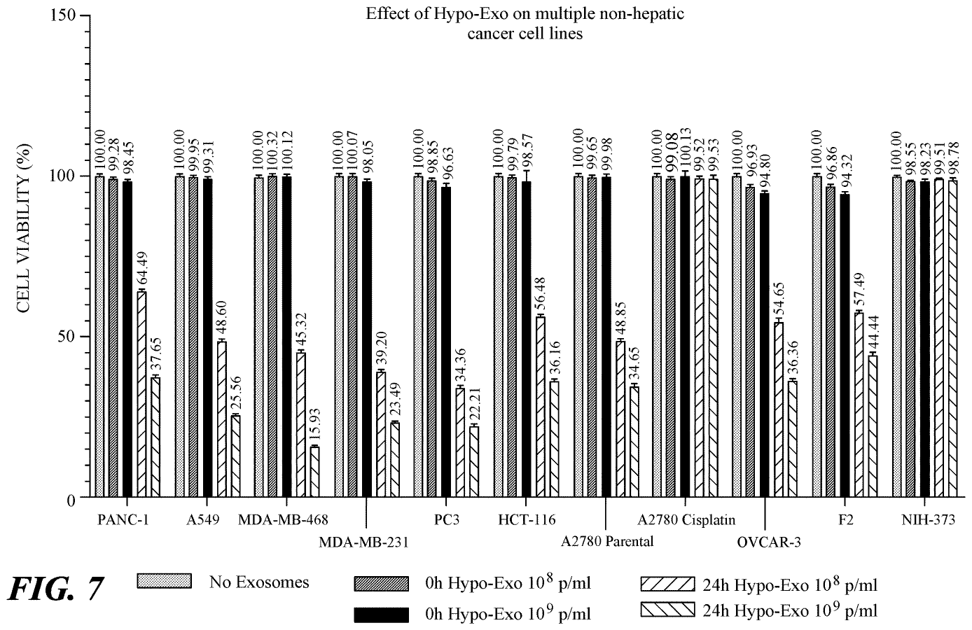

24H-Hypo-Exo were then tested at concentrations of 10⁸ and 10⁹ p/ml for five days against an array of non-hepatic cancers and against the benign NIH 3T3 fibroblast cell line (FIG. 7). 24 h-Hypo-Exo, at a dose of 10⁹ p/ml, caused a decrease in cell viability for PANC-1 (pancreas) of 64%, A549 (lung) of 76%, MDA-MD-468 (breast) of 86%, MDA-MD-231 (breast) of 77%, PC3 (prostate) of 78%, HCT-116 (colon) of 64%, A2780 (ovarian) of 65%, OVCAR (ovarian) of 64% and F2 (ovarian) of 56%. The exosomes again had no activity against the A2780cis (multi-resistant ovarian cancer line) or the benign NIH 3T3 (fibroblast) lines. The data show Hypo-Exo have a potent anti-cancer effect against cancer cells of a wide array of tissue and organ origin while having no effect against benign cells. Again exosomes from HCEC cells not exposed to hypoxia (0h-Hypo-Exo) have no effect on any cell viability.

D. Exosomes Derived from HCEC Cells Transfected with miR-214 and then Challenged with Hypoxia (m214-Hypo-Exo) have Enhanced Activity on Liver and Pancreatic Cancer Cells

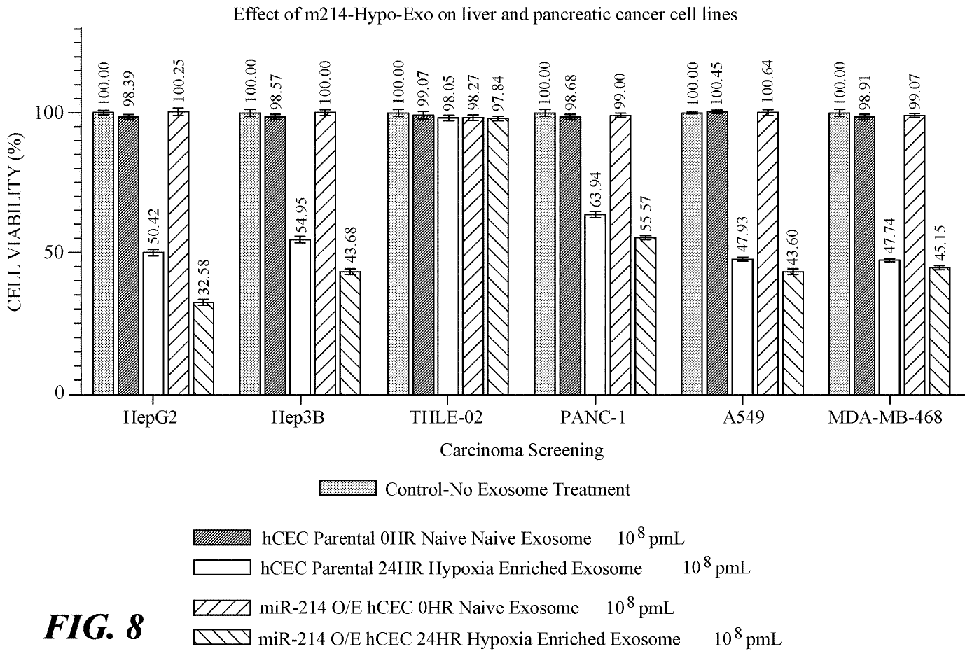

MiR-214 enriched exosomes were generated by transfecting miR-214 encoding pre-RNA into parental HCEC cells. Enrichment of miR-214 in the resulting generated exosomes (HCEC-m214-Exo) was confirmed with RT-PCR (See Methods). The miR-214 expressing HCEC cells were then exposed to hypoxic conditions for 24 hours and exosomes were isolated from the media. The resulting m214-Hypo-Exo were used to treat the HCC cells lines, HepG2 and Hep3B, the pancreatic cancer cell line PANC-1, the lung cell line A549 and the benign liver cell line THLE-2. All exosomes were used at a concentration of 10⁸ p/ml for a total of five days. For the HCC and PANC-1 cancer lines, the miR-214-hypoxia-exosomes were more effective in reducing cell viability than Hypo-Exo without miR-214 enrichment (FIG. 8). For HepG2 cells, the viability was reduced from 50.4% to 32.6%, for Hep3B cells from 55.0% to 43.7% and for PANC-1 cells from 64% to 55.6%. HCEC-m214-exosomes not generated under hypoxic conditions had no impact on cell viability and no exosome population had an effect on the non-cancerous THLE-2 cells.

### E. Hypo-Exo Enhance the Efficacy of the Anti-Cancer Agent, Sorafenib, on Liver Cancer Cells

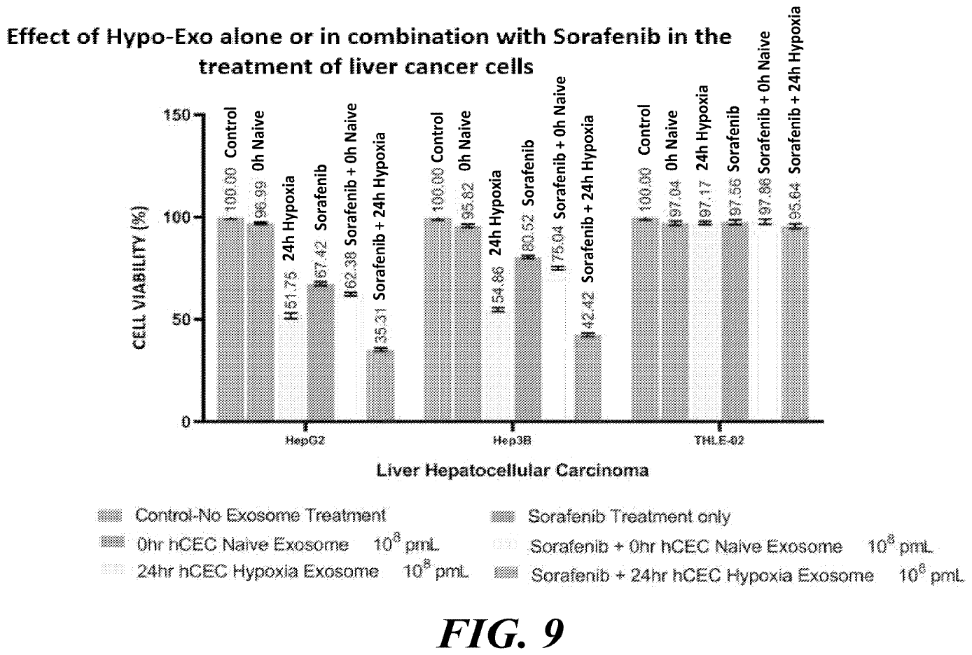

We performed studies to determine if treatment of liver cancer cells with Hypo-Exo and Sorafenib in combination would have additive efficacy. Sorafenib is a small molecule multi-kinase inhibitor approved for therapy of HCC in humans. HepG2 and Hep3B HCC cells were treated with Hypo-Exo alone, Sorafenib alone or the combination of Hypo-Exo and Sorafenib. Sorafenib doses were determined and used that generated incomplete killing of HCC cells and were 1.2 μM for HepG2 and 0.8 UM for Hep3B. Hypo-Exo were used at a concentration of 10⁸ p/ml. There was an additive effect on cell viability when Hypo-Exo were used in combination with Sorafenib (FIG. 9). For HepG2 cells, Hypo-Exo alone reduced cell viability down to 51.8%, Sorafenib alone to 67.4% and the combination to 35.3%. For Hep3B cells, Hypo-Exo alone reduced cell viability to 54.9%, Sorafenib to 80.5% alone and the combination to 42.4%. The addition of non-hypoxic exosomes (0h-Hypo-Exo) to Sorafenib enhanced cytotoxicity by about 5% for both HepG2 and Hep3B cells. Both Sorafenib and Hypo-Exo alone and the combination of the two had negligible impact on benign THLE-2 cells. These data suggest that Hypo-Exo have a major additive effect on the anti-cancer effect of Sorafenib.

F. Exosomes Derived from Multiple Other Parental Cells Challenged by Hypoxia Did not Affect Cancer Cells

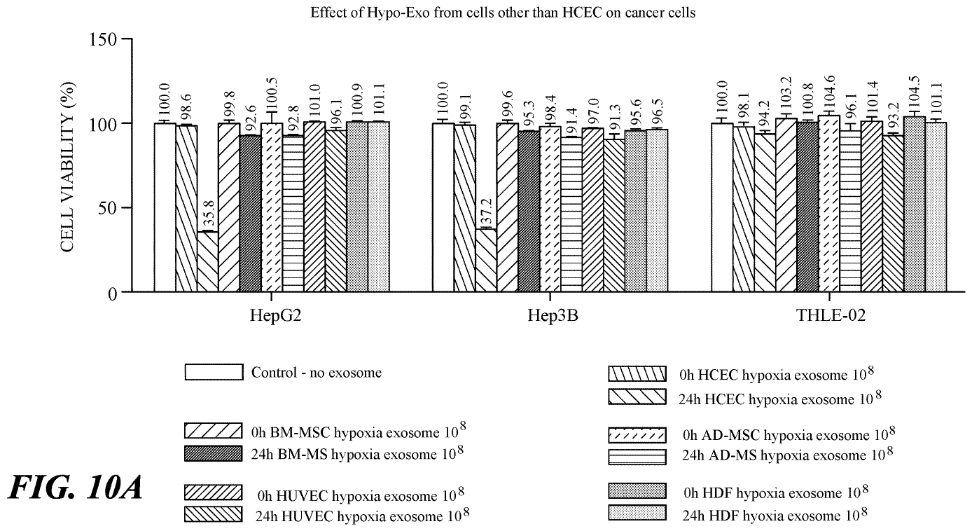

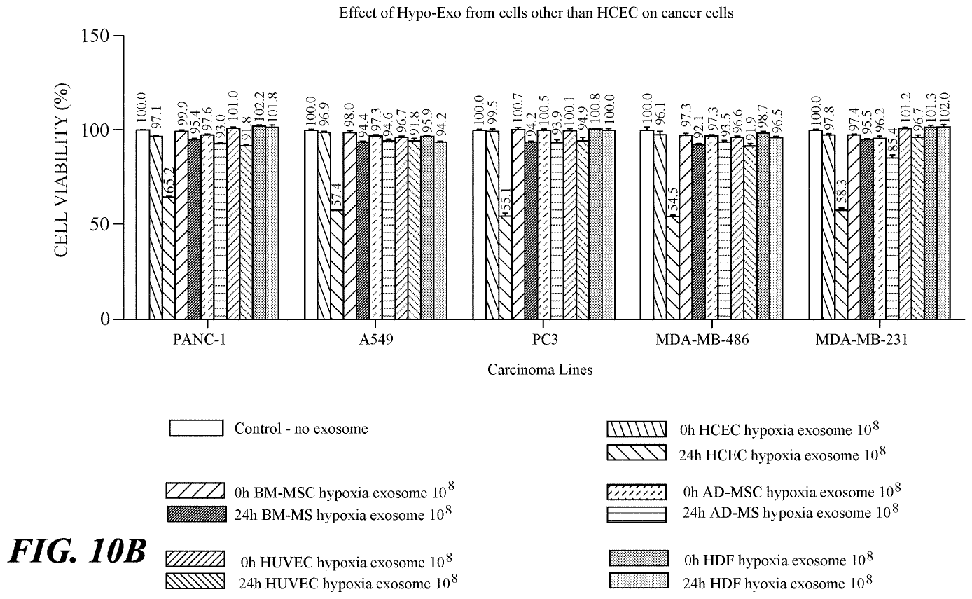

While HCEC cells exposed to hypoxia could generate exosomes toxic to an array of cancer cells, it was not clear if this was an attribute specific to HCEC cells. To help determine if that was the case, we obtained additional human cells and stem cells, exposed them to hypoxia and then tested the exosomes from these cells for anti-cancer activity (FIG. 10). Cells used include bone marrow derived mesenchymal stem cells (BM-MSC), adipose derived mesenchymal stem cells (AD-MSC), primary human umbilical vein endothelial cells (HUVEC) and primary human dermal fibroblast (HDFa) cells (Table 2).

TABLE 2
Additional parental cells used for the generation of Hypo-Exo

| | | | Catalog |
|---|---|---|---|
| Description | Abbreviation | Source (Company) | Number |
| Primary Umbilical Vein | HUVEC | American Type | PCS-100-013 |
| Endothelial Cells; Normal, | | Culture Collection | |
| Human | | (ATCC) | |
| Dermal Fibroblasts; | DFB | American Type | PCS-201-012 |
| Normal, Human, Adult | | Culture Collection | |
| | | (ATCC) | |
| Bone Marrow-Derived | BM-MSC | American Type | PCS-500-012 |
| Mesenchymal Stem Cells. | | Culture Collection | |
| Normal Human | | (ATCC) | |
| Adipose-Derived | Adi-MSC | American Type | PCS-500-011 |
| Mesenchymal Stem Cells. | | Culture Collection | |
| Normal Human | | (ATCC) | |
| Primary Human Cerebral | hCEC | Cell System | ACBRI 376 |
| Endothelial Cells | | | |

We compared exosomes from these cells prior to any hypoxia (0 h) to exosomes obtained after 24 hours of hypoxia (24h). 0 h and 24 h-Hypo-Exo from these new cells were compared to 24 h-Hypo-Exo from HCEC cells to determine their effect on HCC cell lines. Cancer cells were exposed to exosomes at 10⁸ p/ml for five days and cell viability was assessed via the MTT assay. Cancer cells unexposed to exosomes were the control standard. Hypo-Exo from HCEC cells again led to a significant decline in the viability of HepG2 and Hep3B HCC cells with no impact on THLE-2 cells (FIG. 10A). Exosomes from the BM-MSC, AD-MSC, HUVEC and HDFa cell lines had minimal impact (<9%) on any of the HCC cell lines with or without hypoxia.

Hypo-Exo from the non-HCEC cells and HCEC cells were then compared to determine their toxicity against carcinoma cancer cells other than HCC (FIG. 10B). Hypo-Exo were tested against pancreas (PANC-1), lung (A549), prostate (PC3) and breast (MDA-MB-468: MDA-MB-231) cancer cells. Again exosomes from hypoxic HCEC cells demonstrated anti-cancer activity across all cancer cells whereas Hypo-Exo from other cells did not with the exception of a 15% decrease in MDA-MB-231 breast cancer cells when exposed to exosomes from hypoxic AD-MSCs.

These experiments suggest the anti-cancer activity of exosomes from hypoxic cells are primarily confined to HCEC cells of the cells tested.

### G. Mechanisms Underlying the Effect of Stroke-Exos on Anti-Cancer Cells: Hypo-Exo Require Target Cancer Cell Uptake for Anti-Cancer Activity

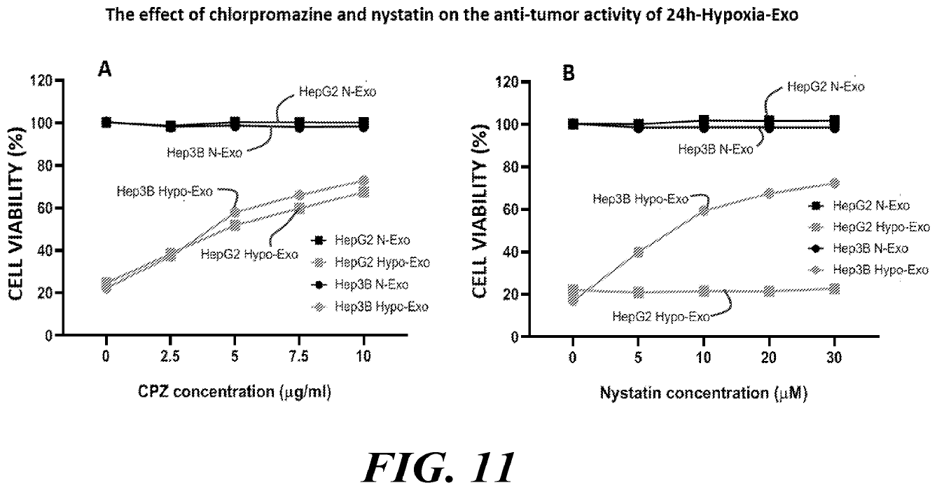

The clathrin and caveolin signaling pathways mediate endocytosis of exosomes. To determine if the anti-cancer activity of Hypo-Exo required their uptake by target HCC cells, we performed experiments with clathrin and caveolin inhibitors. We first tested the effect of chlorpromazine (CPZ), a cell-permeable clathrin inhibitor, or nystatin, an inhibitor of caveolin-dependent endocytosis, on cancer viability. We found that treatment of cancer cells with CPZ at a dose range from 2.5 to 10 μg/ml or nystatin at dose range from 5 to 30 μM (in black) did not affect cancer viability (FIG. 11).

However, the pretreatment of HepG2 and Hep3B with CPZ significantly attenuated the anti-cancer effect of Hypo-Exo on reduction of HCC cell viability in a dose dependent manner (FIG. 11A). The pretreatment of HepG2 and Hep3B with nystatin significantly diminished the anti-cancer effect of Hypo-Exo on the reduction of Hep3B cells in a dose dependent manner, but did not blunt the effect of Hypo-Exo on the reduction of HepG2 cells (FIG. 11B). However, HepG2 cells do not express caveolin and, in theory, would not be affected by caveolin inhibitors. Together, these data provide evidence that Hypo-Exo uptake by Hep3B cells uses both clathrin and caveolin-dependent endocytosis, while Hypo-Exo uptake of HepG2 cells uses clathrin, but not caveolin pathways. The data suggest that the effect of Hypo-Exo on reduction of cancer cells requires exosome internalization.

H. Mechanisms Underlying the Effect of Hypo-Exo on Cancer Cells: Hypo-Exo have a Distinct Protein Profile from Naïve Non-Hypoxia CEC-Exosomes (Naïve-Exo)

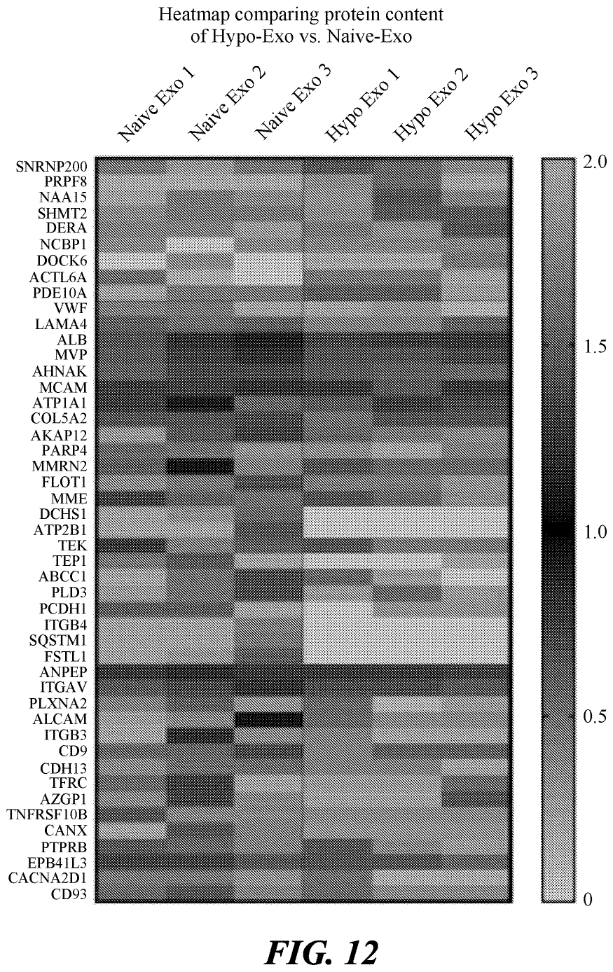

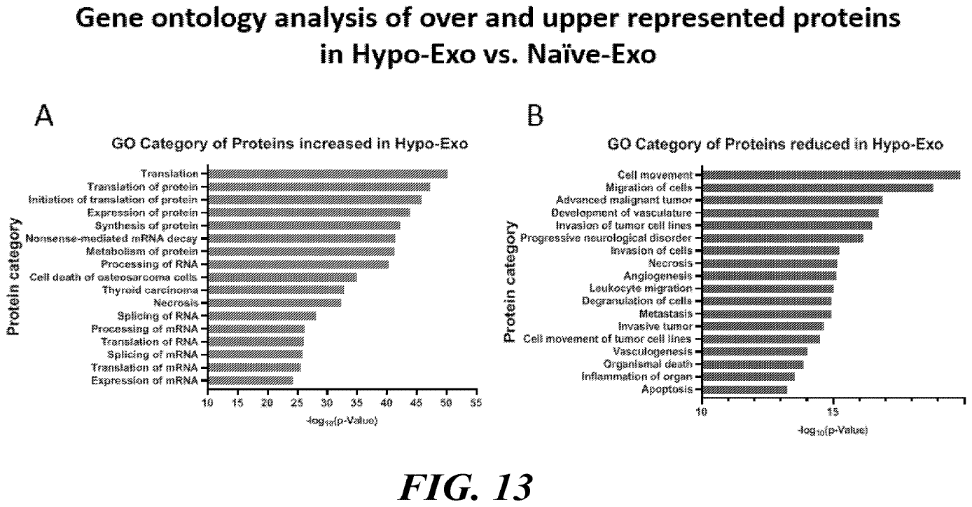

To investigate potential mechanisms underlying the effect of Hypo-Exo on anti-cancer cells, we examined protein profiles in hypoxia-exosomes and naive exosomes by means of Q-Exactive mass spectrometer. Using a threshold of fold change of 0.5 to 1.5 with p value cutoff of 0.05, we found that compared to Naïve-Exo, Hypo-Exo substantially alter protein profiles as shown by the heatmap in FIG. 12. Gene ontology (GO) analysis revealed that many of the top downregulated proteins in Hypo-Exo are associated with cell migration and movement, advanced malignancies and tumor invasion and angiogenesis. (FIG. 13A). Hypo-Exo enriched proteins primarily regulate protein synthesis, metabolism and RNA processing (FIG. 13B). Many proteins were only present in the Hypo-Exo (Table 3).

| | | T-Test |
|---|---|---|
| Alternate | | (p-value): |
| ID | Identified Proteins | (p <0.05) |
| RNMT | mRNA cap guanine-N7 methyltransferase OS = Homo sapiens OX = 9606 | 0.00025 |
| | GN = RNMT PE = 1 SV = 1 | |
| GAR1 | H/ACA ribonucleoprotein complex subunit 1 OS = Homo sapiens | 0.00044 |
| | OX = 9606 GN = GAR1 PE = 1 SV = 1 | |
| MAN2C1 | Alpha-mannosidase 2C1 OS = Homo sapiens OX = 9606 GN = MAN2C1 | 0.00047 |
| | PE = 1 SV = 1 | |
| SERPINF1 | Pigment epithelium-derived factor OS = Homo sapiens OX = 9606 | 0.00059 |
| | GN = SERPINF1 PE = 1 SV = 4 | |
| DDX39A | ATP-dependent RNA helicase DDX39A OS = Homo sapiens OX = 9606 | 0.0006 |
| | GN = DDX39A PE = 1 SV = 2 | |
| MCM3 | DNA replication licensing factor MCM3 OS = Homo sapiens OX = 9606 | 0.0012 |
| | GN = MCM3 PE = 1 SV = 3 | |
| PCID2 | PCI domain-containing protein 2 OS = Homo sapiens OX = 9606 | 0.0013 |
| | GN = PCID2 PE = 1 SV = 2 | |
| PSMG1 | Proteasome assembly chaperone 1 OS = Homo sapiens OX = 9606 | 0.0025 |
| | GN = PSMG1 PE = 1 SV = 1 | |
| SSRP1 | FACT complex subunit SSRP1 OS = Homo sapiens OX = 9606 GN = SSRP1 | 0.0027 |
| | PE = 1 SV = 1 | |
| MVD | Diphosphomevalonate decarboxylase OS = Homo sapiens OX = 9606 | 0.0031 |
| | GN = MVD PE = 1 SV = 1 | |
| RRP9 | U3 small nucleolar RNA-interacting protein 2 OS = Homo sapiens | 0.0065 |
| | OX = 9606 GN = RRP9 PE = 1 SV = 1 | |
| SMARCA5 | SWI/SNF-related matrix-associated actin-dependent regulator of | 0.0079 |
| | chromatin subfamily A member 5 OS = Homo sapiens OX = 9606 | |
| | GN = SMARCA5 PE = 1 SV = 1 | |
| NOP2 | Probable 28S rRNA (cytosine(4447)-C(5))-methyltransferase OS = Homo | 0.0081 |
| | sapiens OX = 9606 GN = NOP2 PE = 1 SV = 2 | |
| TNC | Tenascin OS = Homo sapiens OX = 9606 GN = TNC PE = 1 SV = 3 | 0.0087 |
| PPT1 | Palmitoyl-protein thioesterase 1 OS = Homo sapiens OX = 9606 GN = PPT1 | 0.0092 |
| | PE = 1 SV = 1 | |
| RSL1D1 | Ribosomal L1 domain-containing protein 1 OS = Homo sapiens OX = 9606 | 0.013 |
| | GN = RSL1D1 PE = 1 SV = 3 | |
| DIRAS1 | GTP-binding protein Di-Ras1 OS = Homo sapiens OX = 9606 GN = DIRAS1 | 0.013 |
| | PE = 1 SV = 1 | |
| LRRC40 | Leucine-rich repeat-containing protein 40 OS = Homo sapiens OX = 9606 | 0.014 |
| | GN = LRRC40 PE = 1 SV = 1 | |
| MCM4 | DNA replication licensing factor MCM4 OS = Homo sapiens OX = 9606 | 0.016 |
| | GN = MCM4 PE = 1 SV = 5 | |
| RGN | Regucalcin OS = Homo sapiens OX = 9606 GN = RGN PE = 1 SV = 1 | 0.025 |
| PDS5A | Sister chromatid cohesion protein PDS5 homolog A OS = Homo sapiens | 0.033 |
| | OX = 9606 GN = PDS5A PE = 1 SV = 1 | |
| PDS5B | Sister chromatid cohesion protein PDS5 homolog B OS = Homo sapiens | 0.037 |
| | OX = 9606 GN = PDS5B PE = 1 SV = 1 | |
| GALC | Galactocerebrosidase OS-Homo sapiens OX = 9606 GN = GALC PE = 1 | 0.041 |
| | SV = 3 | |
| NDUFV1 | NADH dehydrogenase [ubiquinone] flavoprotein 1, mitochondrial | 0.042 |
| | OS = Homo sapiens OX = 9606 GN = NDUFVI PE = 1 SV = 4 | |
| PFKFB2 | 6-phosphofructo-2-kinase/fructose-2,6-bisphosphatase 2 OS = Homo | 0.044 |
| | sapiens OX = 9606 GN = PFKFB2 PE = 1 SV = 2 | |
| PPP4C | Serine/threonine-protein phosphatase 4 catalytic subunit OS = Homo | 0.047 |
| | sapiens OX = 9606 GN = PPP4C PE = 1 SV = 1 | |
| MCM2 | DNA replication licensing factor MCM2 OS-Homo sapiens OX = 9606 | 0.05 |
| | GN = MCM2 PE = 1 SV = 4 | |
| MYH14 | Myosin-14 OS = Homo sapiens OX = 9606 GN = MYH14 PE = 1 SV = 2 | <0.00010 |
| ROCK1 | Rho-associated protein kinase 1 OS = Homo sapiens OX = 9606 | <0.00010 |
| | GN = ROCK1 PE = 1 SV = 1 | |
| EMG1 | Ribosomal RNA small subunit methyltransferase NEP1 OS = Homo | <0.00010 |
| | sapiens OX = 9606 GN = EMG1 PE = 1 SV = 4 | |
| MTA1 | Metastasis-associated protein MTA1 OS = Homo sapiens OX = 9606 | <0.00010 |
| | GN = MTA1 PE = 1 SV = 2 | |
| AFP | Alpha-fetoprotein OS = Homo sapiens OX = 9606 GN = AFP PE = 1 SV = 1 | <0.00010 |
| RNF123 | E3 ubiquitin-protein ligase RNF123 OS = Homo sapiens OX = 9606 | <0.00010 |
| | GN = RNF123 PE = 1 SV = 1 | |

These data suggest that altered cargo proteins in Hypo-Exo could be used as a specific signature, which mediates the observed anti-cancer cell effect of Hypo-Exo.

I. Mechanisms Underlying the Effect of Hypo-Exo on Cancer Cells: Hypo-Exo have a Distinct miRNA Profile from Naïve-Exo

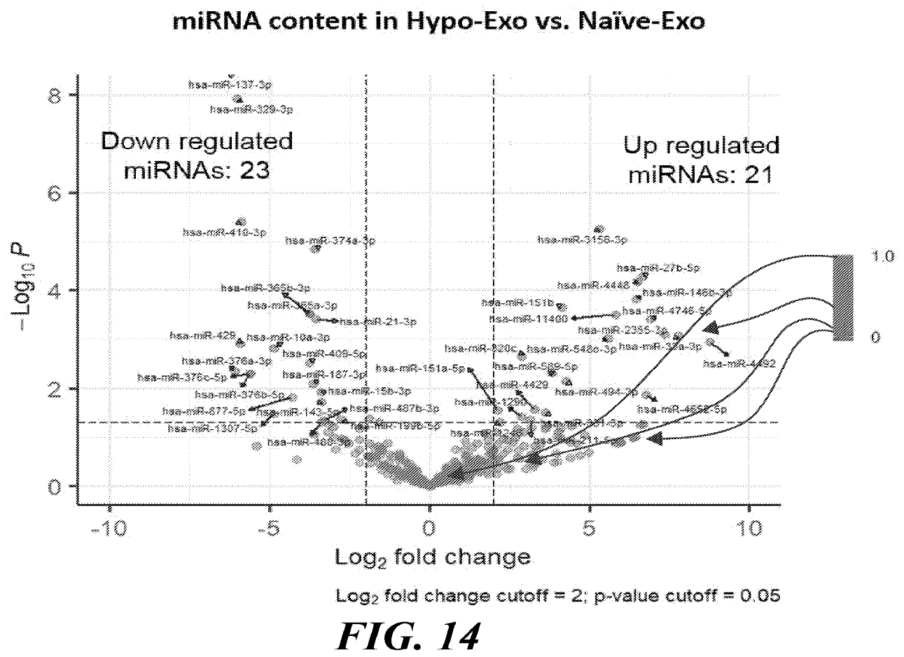

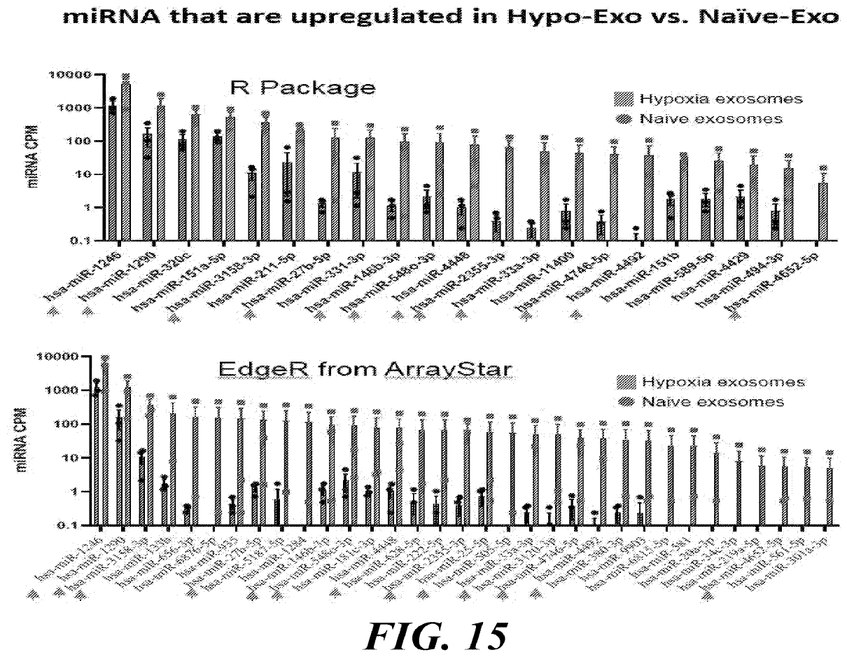

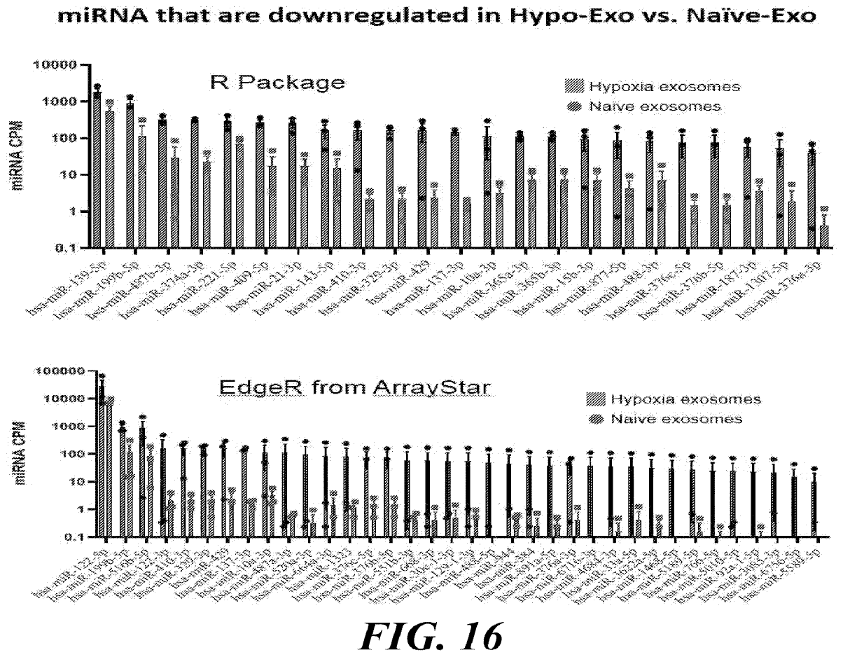

To investigate mechanisms underlying the activity of Hypo-Exo on cancer cells, we compared the RNA content of Hypo-Exo to non-Hypo-Exo or Naïve-Exo from HCEC cells. RNA content was determined by RNA-seq using next generation sequencing and both the EdgeR (ArrayStar) and R package were used for statistical analysis. Both volcano plots (FIG. 14) and bar plots were generated showing miRNA that are upregulated (FIG. 15) or downregulated (FIG. 16) in Hypo-Exo compared to Naïve-Exo. In the volcano plot (FIG. 14), a significant difference in miRNA expression is defined as a >2 fold log difference with a p-value cutoff of 0.05. The bar graphs compare findings using the two different statistical products.

Several enriched miRNAs in Hypo-Exo include miR-146b and miR-27b which are known to suppress tumor cell growth (Katakowski). Moreover, there were several miRNAs that were detected only within Hypo-Exo (Table 4).

TABLE 4
miRNAs only presented in Hypoxia-Exo.

| Mature_ID | Mature_Seq | Seed | log2FC | Fold_Change | p_value |
|---|---|---|---|---|---|
| hsa-miR-6876-5p | CAGGAAGGAGACAGGCA | AGGAAG | 11.755468 | 3457.3907 | 0.0008046 |
| | GUUCA (SEQ ID NO: 2) | | | | |
| hsa-miR-1284 | UCUAUACAGACCCUGGC | CUAUAC | 11.342762 | 2597.2367 | 0.0017161 |
| | UUUUC (SEQ ID NO: 3) | | | | |
| hsa-miR-505-5p | GGGAGCCAGGAAGUAUU | GGAGCC | 10.297459 | 1258.4732 | 0.0080691 |
| | GAUGU (SEQ ID NO: 4) | | | | |
| hsa-miR-6815-5p | UAGGUGGCGCCGGAGGA | AGGUGG | 9.0394241 | 526.18419 | 0.0242078 |
| | GUCAUU (SEQ ID NO: 5) | | | | |
| hsa-miR-581-5p | UCUUGUGUUCUCUAGAU | CUUGUG | 9.0030983 | 513.10075 | 0.0246918 |
| | CAGU (SEQ ID NO: 6) | | | | |
| hsa-miR-20a-3p | ACUGCAUUAUGAGCACU | CUGCAU | 8.3544496 | 327.29541 | 0.0334271 |
| | UAAAG (SEQ ID NO: 7) | | | | |
| hsa-miR-4492-5p | GGGGCUGGGCGCGCGCC | GGGCUG | 8.1891858 | 291.87073 | 0.0255536 |
| | (SEQ ID NO: 8) | | | | |
| hsa-miR-34c-3p | AAUCACUAACCACACGG | AUCACU | 7.5323611 | 185.12566 | 0.0360981 |
| | CCAGG (SEQ ID NO: 9) | | | | |
| hsa-miR-219a-5p | UGAUUGUCCAAACGCAA | GAUUGU | 7.081145 | 135.40573 | 0.0430224 |
| | UUCU (SEQ ID NO: 10) | | | | |
| hsa-miR-4652-5p | AGGGGACUGGUUAAUAG | GGGGAC | 7.0366988 | 131.29779 | 0.0436499 |
| | AACUA (SEQ ID NO: 11) | | | | |
| hsa-miR-561-5p | AUCAAGGAUCUUAAACU | UCAAGG | 6.9488446 | 123.54087 | 0.0460648 |
| | UUGCC (SEQ ID NO: 12) | | | | |
| hsa-miR-301a-3p | CAGUGCAAUAGUAUUGU | AGUGCA | 6.874665 | 117.34926 | 0.0477887 |
| | CAAAGC (SEQ ID NO: 13) | | | | |

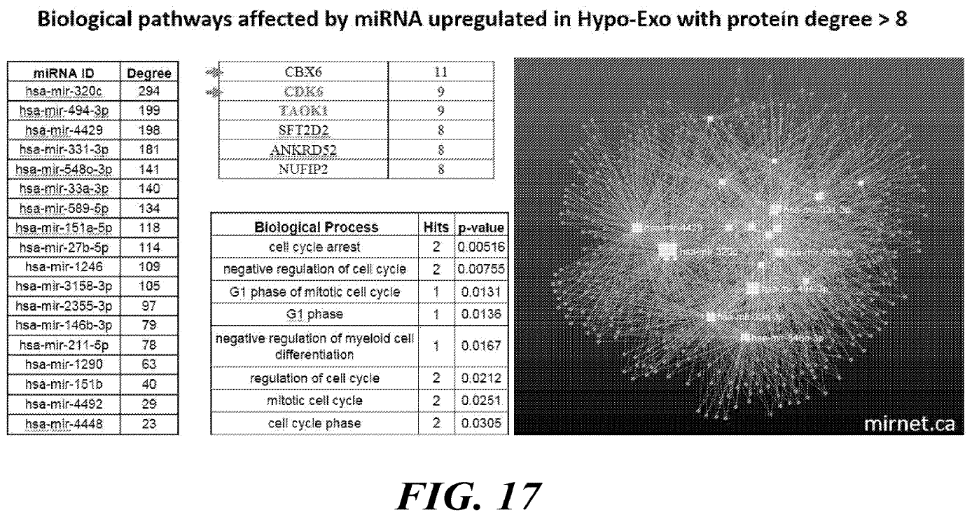

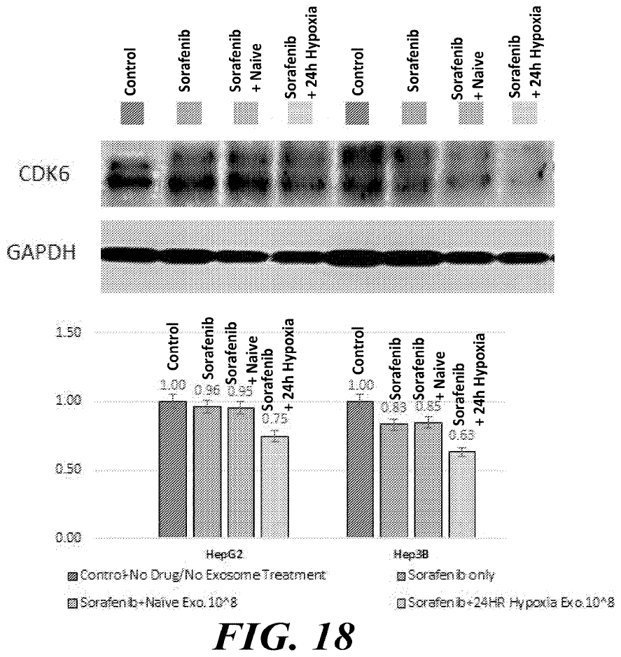

Thus, these miRNAs can potentially target genes that mediate cancer progression. For example, we found that there were nine miRNAs enriched in Hypo-Exo that can directly target CDK6 (FIG. 17). CDK6 is a cell cycle regulatory protein. CDK6 Inhibitors have been clinically used for cancer therapy. Our data showed that treatment of HepG2 and Hep3B cells with Hypo-Exo in combination with Sorafenib robustly reduced CDK6 (FIG. 18), suggesting that enriched miRNAs in Hypo-Exo can play a role in mediating the anti-cancer activity of Hypo-Exo.

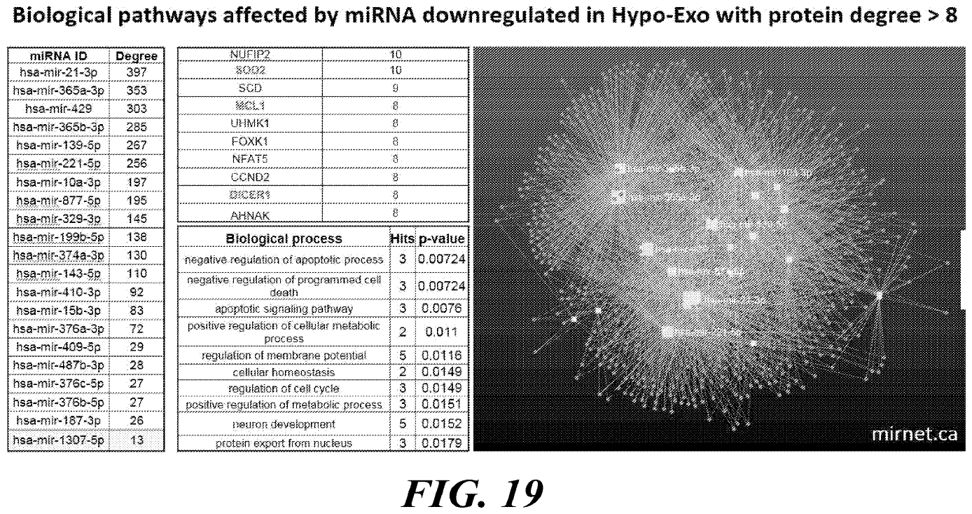

The differential expression of miRNA was then analyzed using pathway analysis to identify protein-protein interactions with a degree >8 and a p-value <0.05. The analysis demonstrated that the primary pathways affected by upregulated miRNA in hypoxia-exosomes mediate cell-cycle arrest and negative regulation of the cell-cycle in general (FIG. 17). The analysis demonstrated that the primary pathways affected by downregulated miRNA (FIG. 19) in hypoxia-exosomes negatively regulated apoptosis and programmed cell death. In toto, these data indicate that hypoxia-exosomes induce activity against cancerous cells, in part, through pathways of cell cycle arrest, apoptosis and programmed cell death.

### Materials and Methods

Cancer cell lines used in the present study are listed in Table 1, while Table 2 lists human cells used for exosome isolation.

Generation of OGD-Exo: Primary human primary cerebral endothelial cells (HCEC) were cultured in endothelial medium (Cell Systems, 4Z0-500) until the cells reached 80-90% confluence. HCEC were then subjected to glucose-free DMEM (Gibco) medium and placed in an oxygen-free chamber for indicated times. After rinsing with PBS, HCEC were cultured in serum-free medium (SF-4Z0-500). Conditioned medium was collected every 2-3 days for one week. Exosomes were isolated from conditioned medium by the differential ultracentrifugation method.

Generation of Hypoxia-Exo: HCEC were grown in endothelial medium (Cell Systems, 4Z0-500) until the cells reached 80-90% confluence. HCEC were then subjected to oxygen-free conditions in an oxygen-free chamber for indicated times. Cells were rinsed with PBS after treatment and grown in serum-free medium (SF-4Z0-500). Conditioned medium was collected every 2-3 days for one week. Exosomes were isolated from conditioned medium using the differential ultracentrifugation method.

Generation of CEC-m214-Exo: HCEC were transfected with the human pre-microRNA expression construct Lenti-miR-214 (PMIRH214PA-1, System Biosciences). The transfected HCEC were cultured in endothelial medium for 48 h and then cultured in serum-free medium for another 48h. Conditioned medium was collected every 2-3 days for one week. Exosomes were isolated from conditioned medium by the differential ultracentrifugation method.

All exosomes were characterized by nanoparticle tracking analysis (NTA) system, transmission electron microscopy (TEM) and Western blot analysis for exosome marker proteins. Quantitative RT-PCR analysis was performed to measured miR-214 levels in HCEC and CEC-m214-Exo.

3-(4,5-dimethylthiazol-2-yl)-2,5 diplenyltetrazolium bromide (MTT) assay: To measure cell viability, cells were seeded in 96-well plates at a density of 800 cells per well. Cells were cultured in medium with indicated treatment. After 5 days of treatment, medium was removed and MTT was added to each well with an additional 4 hr incubation to allow mitochondrial dehydrogenase to convert MTT into insoluble formazan crystals. The absorption of solubilized formazan was measured at a wavelength of 490 nm by an ELISA plate reader (EL340 microplate reader; Bio-Tek Instruments, Winooske, VT).

### REFERENCES

Lovet J M, Ricci S, Mazzaferro V, Hilgard P, Gane E, Blanc J F, Cosme de Oliveira A, Santoro A, Raoul J L, Forner A, Schwartz M, Porta C, et al. Sorafenib in advanced hepatocellular carcinoma. N Engl J Med. 2008; 359:378-390.
Semaan L, Zeng Q, Lu Y, Zhang Y, Zreik M M, Chamseddine M B, Chopp M, Zhang, Z G, and Moonka D. MicroRNA-214 enriched exosomes from human cerebral endothelial cells (hCEC) sensitize hepatocellular carcinoma to anti-cancer drugs. Oncotarget. 2021 Vol. 12, pp: 185-198
Doyle L M and Wang M Z. Overview of extracellular vesicles, their origin, composition, purpose, and methods of isolation and analysis. Cells 2019 8; 7:727
Kalluri R and V S LeBleu. The biology, function, and biomedical applications of exosomes. Science 2020 367; 6478:
Katakowski M, Buller B, Zheng X, Lu Y, Rogers T, Osobamiro O, Shu W, Jiang F, Chopp M. Exosomes from marrow stromal cells expressing miR-146b inhibit glioma growth. Cancer Lett. 2013; 335:201-4.

While some embodiments have been particularly shown and described with reference to the foregoing preferred and alternative embodiments, it should be understood by those skilled in the art that various alternatives to the embodiments described herein may be employed in practicing the invention without departing from the spirit and scope of the invention as defined in the following claims. It is intended that the following claims define the scope of the invention and that the methods, systems, and compositions within the scope of these claims and their equivalents be covered thereby.

This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A method for preventing or treating a cancer, the method comprising administering a therapeutically effective amount of stressed mammalian extracellular vesicles derived from hypoxia or oxygen and glucose deprived mammalian cells, to a subject in need thereof.

2. The method of claim 1, wherein the stressed mammalian extracellular vesicles are stressed mammalian exosomes.

3. The method of claim 1 or 2, wherein the cancer to be treated or prevented comprises liver cancer or pancreatic cancer.

4. The method of claim 1 or 2, wherein the cancer is hepatocellular carcinoma, prostate cancer, lung cancer, breast cancer, ovarian cancer, or colon cancer.

5. The method according to any one of claims 1 to 4, wherein the stressed mammalian extracellular vesicles are combined with exosomes that are enriched with miR-214.

6. The method according to any one of claims 2 to 5, wherein the stressed mammalian exosomes comprise the markers Alix and CD63.

7. The method according to any one of claims 2 to 6, wherein the stressed mammalian exosomes are derived and isolated from stressed cerebral endothelial cells (CECs), brain microvascular endothelial cells (BMVEC), or Primary Human Brain Microvascular Endothelial Cells (PHBMVEC).

8. The method according to any one of claims 2 to 7, wherein the stressed mammalian exosomes are derived and isolated from stressed cerebral endothelial cells (CECs).

9. The method according to any one of claims 2 to 8, wherein the stressed mammalian exosomes are derived from hypoxia stressed mammalian cells.

10. The method according to any one of claims 2 to 8, wherein the stressed mammalian exosomes are derived from oxygen and glucose (OGD) stressed mammalian cells.

11. The method according to any one of claims 2 to 10, wherein the subject is administered about 1×10¹ to about 1×10¹⁹ stressed mammalian exosomes per kg body weight of the subject.

12. The method according to any one of claims 2 to 10, wherein the subject is administered about 1×10⁹ to about 1×10¹⁵ exosomes per kg body weight of the subject.

13. The method according to any one of claims 2 to 10, wherein the subject is administered about 1×10⁵ to about 1×10¹⁹ stressed exosomes per dose, or per daily dose, administered intravenously (IV), subcutaneously (S.C.), or orally to the subject with cancer.

14. The method according to any one of claims 2 to 13, wherein the stressed mammalian exosomes are autologous or allogeneic.

15. The method according to any one of claims 2 to 14, wherein the exosomes are derived from human primary tissue brain endothelial cells or from human brain tissue cultured cells.

16. The method according to any one of claims 2 to 15, wherein the exosomes comprise miR-214.

17. The method according to any one of claims 1 to 16, wherein the subject is further administered one or more anti-cancer agents, sequentially or concomitantly, with the stressed mammalian extracellular vesicles derived from hypoxia or oxygen and glucose deprived mammalian cells.

18. The method according to claim 17, wherein the anti-cancer agent is a chemotherapeutic agent comprising: platinum agents, taxanes, vinca alkaloids, boronic acid derivatives, an EGFR or ErbB inhibitor, a VEGFR inhibitor, a PARP inhibitor, phthaloyl derivatives, or epothilones.

19. The method according to claim 17 or 18, wherein the anti-cancer agent comprises sorafenib or oxaliplatin.

20. A method of treating cancer in a subject in need thereof, the method comprising administering a therapeutically effective amount of stressed mammalian exosomes in combination with one or more chemotherapeutic agents.

21. The method according to claim 20, wherein the stressed mammalian exosomes are derived from endothelial cells, endothelial progenitor cells, cerebral endothelial cells (CEC), AG-133/CD-133+ cells, Schwann cells, glial cells, astrocytes, neurons, oligodendrocytes, spindle neurons, microglia, mastocytes, or combinations thereof.

22. The method according to claim 20 or 21, wherein the stressed mammalian exosomes comprise miR-214.

23. The method according to any one of claims 20 to 22, wherein the subject is dosed with a therapeutically effective amount of stressed mammalian exosomes in an amount ranging from about 1×10¹ to about 1×10¹⁹ exosomes per kg body weight of the subject.

24. The method according to claim 23, wherein the subject is administered about 1×10⁹ to about 1×10¹⁵ stressed mammalian exosomes per kg body weight of the subject.

25. The method according to any one of claims 20 to 24, wherein the subject is administered stressed CEC derived exosomes.

26. The method according to any one of claims 20 to 25, wherein the stressed mammalian exosomes are administered in combination with a chemotherapeutic agent, the chemotherapeutic agent comprising an alkylating agent, an anti-metabolite, an antitumor antibiotic, an antimitotic agent, a topoisomerase I or topoisomerase II inhibitor, an antitumor hormone, a hormonal analog, a poly ADP ribose polymerase (PARP) inhibitor, a retinoid, a signal transduction pathway inhibitor, an EGFR or ErbB inhibitor, a cell growth inhibitor, a growth factor function inhibitor, an angiogenesis inhibitor, a serine/threonine inhibitor, a kinase inhibitor, a cyclin dependent kinase inhibitor, an antisense therapeutic, an immunotherapeutic agent, a cancer vaccine, or combinations thereof.

27. The method according to any one of claims 20-26, wherein the chemotherapeutic agent comprises sorafenib or oxaliplatin.

28. The method according to any one of claims 20 to 27, wherein the exosomes are administered prior to, concurrently with, or subsequent to the administration of the chemotherapeutic agent.

29. The method according to any one of claims 20 to 28, wherein the combination of the stressed mammalian exosomes and the chemotherapeutic agent are administered in separate compositions.

30. The method according to any one of claims 20 to 28, wherein the combination of the stressed mammalian exosomes and the chemotherapeutic agent are administered in in a single composition.

31. The method according to any one of claims 20 to 30, wherein the cancer is colon carcinoma, breast cancer, lung cancer, pancreatic cancer, hepatocellular carcinoma, or ovarian carcinoma.

32. The method according to any one of claims 20 to 31, wherein the chemotherapeutic agent is dosed at a concentration that is lower than the standard therapeutic dosage for administering the chemotherapeutic agent to a similar subject based on a similar weight, age, type and stage of cancer in the absence of the combination.

33. The method according to any one of claims 20 to 31, wherein the chemotherapeutic agent is dosed at a concentration that is higher than the standard therapeutic dosage for administering the chemotherapeutic agent to a similar subject based on a similar weight, age, type and stage of cancer in the absence of the combination.

34. The method according to any one of claims 20 to 33, wherein administration of the combination reduces, ameliorates, or eliminates one or more side effects associated with multiple administrations of the chemotherapeutic agent alone in a population of similar subjects treated for the same or similar cancer.

35. A stressed mammalian cell derived extracellular vesicle derived from a cell stressed under hypoxic conditions or oxygen and glucose deprivation.

36. The stressed mammalian cell derived extracellular vesicle according to claim 35, wherein the extracellular vesicle is an exosome.

37. The stressed mammalian cell derived extracellular vesicle according to claim 36, wherein the stressed mammalian exosome is enriched with miR-19a, miR-21, miR-27b, miR-146a, miR-146b, or a combination thereof.

38. The stressed mammalian cell derived extracellular vesicle according to claim 36, wherein the stressed mammalian exosome is derived from cerebral endothelial cells (CEC), brain microvascular endothelial cells (BMVEC), Primary Human Brain Microvascular Endothelial Cells (PHBMVEC), endothelial progenitor cells, AG-133/CD-133+ cells, Schwann cells, glial cells, astrocytes, neurons, oligodendrocytes, spindle neurons, microglia, mastocytes or combinations thereof.

39. The stressed mammalian cell derived extracellular vesicle according to claim 38, wherein the stressed mammalian exosome is derived from CECs.

40. The stressed mammalian cell derived extracellular vesicle according to claim 38, wherein the stressed mammalian exosome comprises miR-214.

41. The stressed mammalian cell derived extracellular vesicle according to claim 40, wherein the stressed mammalian exosome is oxygen and glucose deprived and combined with exosomes from enriched mi-214 human cerebral endothelial cells.

42. The stressed mammalian cell derived extracellular vesicle according to claim 38, wherein the stressed mammalian exosome comprises at least two or more of hsa-miR-6876-5p, hsa-miR-1284, hsa-miR-505-5p, hsa-miR-6815-5p, hsa-miR-581-5p, hsa-miR-20a-3p, hsa-miR-4492-5p, hsa-miR-34c-3p, hsa-miR-219a-5p, hsa-miR-4652-5p, hsa-miR-561-5p, and hsa-miR-301a-3p.

43. The stressed mammalian cell derived extracellular vesicle according to claim 38, wherein the stressed mammalian exosome comprises at least three or more of hsa-miR-6876-5p, hsa-miR-1284, hsa-miR-505-5p, hsa-miR-6815-5p, hsa-miR-581-5p, hsa-miR-20a-3p, hsa-miR-4492-5p, hsa-miR-34c-3p, hsa-miR-219a-5p, hsa-miR-4652-5p, hsa-miR-561-5p, and hsa-miR-301a-3p.

44. The stressed mammalian cell derived extracellular vesicle according to any one of claim 42 or 43, wherein the stressed mammalian exosome further comprises miR-214.

45. A pharmaceutical composition comprising the stressed mammalian cell derived extracellular vesicle according to any one of claims 35 to 44, and at least one pharmaceutically acceptable excipient.

46. Use of a composition comprising a stressed mammalian cell derived extracellular vesicle according to any one of claims 35 to 44 for the manufacture of a medicament for the treatment of cancer.

47. The use according to claim 46, wherein the cancer comprises hepatocellular carcinoma, prostate cancer, pancreatic cancer, lung cancer, breast cancer, ovarian cancer, or colon cancer.

48. The use according to any one of claims 46 to 47, wherein the stressed mammalian cell comprises cerebral endothelial cells (CEC), brain microvascular endothelial cells (BMVEC), Primary Human Brain Microvascular Endothelial Cells (PHBMVEC), endothelial progenitor cells, AG-133/CD-133+ cells, Schwann cells, glial cells, astrocytes, neurons, oligodendrocytes, spindle neurons, microglia, mastocytes or combinations thereof.

49. The use according to any one of claims 46 to 48, wherein the stressed mammalian cell comprises human CECs or human endothelial progenitor cells.

50. A kit comprising stressed mammalian exosomes, a dose of a chemotherapeutic agent, and a package insert comprising instructions for using the stressed mammalian exosomes and the chemotherapeutic agent in combination to treat a subject with cancer.
